(19) United States
(12) Patent Application Publication
(10) Pub. No.: US 2023/0364228 A1
(43) Pub. Date: Nov. 16, 2023

(54) METHOD OF TREATING AMYLOIDOSIS

(71) Applicant: Caelum Biosciences, Inc., Bordentown, NJ (US)

(72) Inventors: Michael Spector, Shamong, NJ (US); Susan B. Sobolov-Jaynes, Hamden, CT (US); Michael Kahsai, Flemington, NJ (US); Anthony S. Lubiniecki, West Chester, PA (US); Jessica G. Raviwong, Bridgewater, NJ (US)

(21) Appl. No.: 18/026,185

(22) PCT Filed: Sep. 14, 2021
(86) PCT No.: PCT/US2021/050316

§ 371 Date: Mar. 14, 2023

Related U.S. Application Data
(60) Provisional application No. 63/078,258, filed on Sep. 14, 2020.
(60) Provisional application No. 63/116,100, filed on Nov. 19, 2020.
(60) Provisional application No. 63/176,015, filed on Apr. 16, 2021.

(51) Int. Cl. A61K 39/395; C07K 16/18; A61K 31/675; A61K 31/69; A61K 31/573; A61K 31/198; A61K 31/454; A61K 31/635; A61K 35/545; A61K 47/12; A61K 47/26; A61P 7/00

(52) U.S. Cl. CPC A61K 39/3955; C07K 16/18; A61K 31/675; A61K 31/69; A61K 31/573; A61K 31/198; A61K 31/454; A61K 31/635; A61K 35/545; A61K 47/12; A61K 39/39591; A61K 47/26; A61P 7/00; A61K 2039/545

(57) ABSTRACT

Provided herein are methods of treating amyloidosis disease and disorder in a subject, and methods of decreasing the amount of amyloid deposits in an organ. The methods provided herein include the administration of an antibody, alone or in combination with another therapy. In one aspect, the disclosure provides a method of treating an amyloidosis disease or disorder in a subject including administering to the subject the pharmaceutical composition of the invention and an additional therapy, thereby treating the amyloidosis disease or disorder in the subject.

### CROSS REFERENCE TO RELATED APPLICATIONS

This application claims the benefit of U.S. Provisional Patent Application Nos. 63/176,015, filed Apr. 16, 2021, 63/116,100, filed Nov. 19, 2020 and 63/078,258, filed Sep. 14, 2020 which are herein incorporated by reference in their entirety.

### INCORPORATION OF SEQUENCE LISTING

The material in the accompanying sequence listing is hereby incorporated by reference into this application. The accompanying sequence listing text file, name CAEL2000_3WO_SL.txt was created on Aug. 24, 2021, and is 10 kb. The file can be assessed using Microsoft Word on a computer that uses Window OS.

### BACKGROUND OF THE INVENTION

### Field of the Invention

The present invention relates generally to amyloidosis, and more specifically to methods of treating amyloidosis diseases and disorders using antibodies that bind to mis-folded light chains.

### Background Information

Amyloidosis, or amyloid disease is a rare disease that occurs when amyloid builds up in organs and interferes with their normal function. Amyloid is not normally found in the body, but it can be formed from several different types of protein. Organs that may be affected include the heart, kidneys, liver, spleen, nervous system, skin and digestive tract. Amyloid light-chain amyloidosis (AL amyloidosis, AL, or ALA), also called primary amyloidosis, is the most common form of systemic amyloidosis in the United States and accounts for approximately 70% of the diagnosed cases of amyloidosis in developed countries. However, the term “amyloidosis” refers to a cluster of diseases which share a common feature, i.e., the extracellular deposition of pathologic insoluble fibrillar proteins in organs and tissues. AL amyloidosis is caused by malfunction of antibody-producing cells causing production of abnormal protein fibers that aggregate to form insoluble amyloid deposits in organs and tissues. In primary amyloidosis, the fibrils include intact fragments of immunoglobulin light chains or fragment of light chains, and in secondary amyloidosis, the fibrils include amyloid A protein. Modern classification of amyloidosis is based on the nature of the precursor plasma proteins that form the fibril deposit.

The precursor plasma proteins are diverse and unrelated. Nevertheless, all precursor proteins produce amyloids that are characterized by a fibrillar morphology of 7-13 nm in diameter, a p-sheet secondary structure and ability to be stained by particular dyes, such as Congo red. The final stage in the development of amyloidosis is the deposit of amyloid fibrils in the organs of the patient. Amyloidosis mortality is high, with current five-year survival rates of about 28%.

The most effective treatment for AL amyloidosis is autologous bone marrow transplant with stem cell rescue. For patients ineligible for stem cell transplantation, targeted chemotherapies to eradicate the underlying plasma-cell dyscrasia (PCD) and stop the production of misfolded light chains are used. Such treatments rely on conventional or high dose cytotoxic chemotherapy, such as used in multiple myeloma (e.g., combination of melphalan and dexamethasone, and a combination of bortezomib and dexamethasone). However, since the fibrillar deposits are often asymptomatic until after significant deposition has taken place, cytotoxic chemotherapy is unlikely to be undertaken before significant deposits have already occurred. Additionally, since cytotoxic chemotherapy is, at best, effective only to stop the further production of precursor abnormal protein but not to remove the existing deposits, prognosis for amyloidosis patients remains exceedingly poor due to persistence (or progression) of the pathologic deposits and the absence of improvement and/or reversing of organ dysfunction.

B-cell lymphoproliferative disorders can be associated with systemic AL amyloidosis. AL amyloidosis associated with lymphoproliferative disorders appears to be caused by monoclonal immunoglobulin light chains produced by the neoplastic B-cells that arise in these disorders. Patients with systemic amyloidosis have high levels of M-protein, multiorgan involvement with frequent cardiac involvement, and nephrotic syndrome. Among the lymphoproliferative disorders that have been described that cause Ig monoclonal gammopathies are Waldenstrom's macroglobulinemia (also named lymphoplasmacytic lymphoma), chronic lymphocytic leukemia, and other lymphomas associated with amyloidosis (types of Non-Hodgkin lymphoma).

Native antibodies are immunoglobulin molecules that contain an antigen binding site that bind immune specifically to antigens. Native antibodies are usually heterotetrameric glycoproteins composed of two identical light (L) chains and two identical heavy (H) chains. The light chains can be assigned to kappa (κ) and lambda (λ) subgroups, based on the amino acid sequences of their constant domains. Depending on the amino acid sequence of the constant domain of their heavy chains, immunoglobulins can be an IgA, IgD, IgE, IgG, or IgM. A chimeric antibody is a genetically chimeric heterodimer in which the constant regions of the heavy and light chains are derived from one species (usually human or non-human primate) and the variable regions of the heavy and light chains are of a different species (typically murine).

A mouse-human chimeric immunoglobulin G1 of κ isotype that reacts specifically with human light chain amyloid fibrils, irrespective of their κ or λ isotype but, notably, not with the native forms of kappa or lambda light chain has been developed. The antibody has been shown to bind to a cryptic epitope at the N-terminal of light chain proteins that adopt a non-native β-sheet structure, which is conserved in κ and λ mis-folded light chains. Therapeutic targeting and clearance of amyloid deposits is an area of intense medical interest, for which there is still no approved therapy, and therefore a significant unmet medical need. The compositions and methods disclosed herein fulfill this need.

### SUMMARY

The present disclosure is based on the discovery that the antibody described herein, alone or in combination with an additional therapy, is efficient for the treatment of amyloid deposits, such as amyloidosis diseases and disorders including amyloidosis deposits in patients having a plasma cell disease or disorder. The present invention is exemplified but not limited by the disclosure in the Examples herein.

In one embodiment, the disclosure provides a pharmaceutical composition including an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains (also known as CAEL101); one or more isotonic agents, a buffer and a non-ionic surfactant.

In one aspect, the antibody binds to kappa and lambda mis-folded light chains. In another aspect, the composition includes the antibody, sodium acetate, sodium chloride, mannitol and polysorbate 80. In some aspects, the antibody includes a mixture of antibody molecules including a native fraction, a reduced fraction, and a glycosylated or deglycosylated fraction, any of which having a heterogeneous charge. In other aspects, the antibody includes a mixture including intact antibodies, halfmer fragments, incomplete antibody fragments, other fragments and/or aggregates thereof.

In another embodiment, the disclosure provides a method of decreasing an amount of amyloid deposits in a subject including administering to the subject about 1,000 mg/m² of an antibody having a heavy chain variable domain (VH) having an amino acid sequence of SEQ ID NO:1 and a light chain variable domain (VL) having an amino acid sequence of SEQ ID NO 2.

In one aspect, the antibody is administered weekly for at least 2, 3 or 4 weeks. In other aspects, a maintenance dose of antibody is further administered to the subject thereafter. In various aspects, the maintenance dose is administered biweekly, triweekly, or monthly after the first 2, 3, 4 or more weeks. In one aspect, the subject is newly diagnosed with an amyloidosis disease or disorder prior to administration of the antibody. In other aspects, the subject has been previously treated for an amyloidosis disease prior to administration of the antibody. In one aspect, the amyloidosis disease or disorder is selected from the group consisting of light chain (AL) amyloidosis, autoimmune (AA) amyloidosis and hereditary (TTR) amyloidosis.

In an additional embodiment, the disclosure provides a method of treating an amyloidosis disease or disorder in a subject including administering to the subject the pharmaceutical composition of the disclosure and an additional therapy.

In one aspect, the antibody administration dose is from about 500 mg/m² to 1,000 mg/m². In many aspects, the dose is selected from about 500 mg/m², about 750 mg/m² and about 1,000 mg/m². In one aspect, a weekly dose of about 500 mg/m² of the antibody include about 12.5 mg/kg of antibody, a weekly dose of about 750 mg/m² of the antibody includes about 18.75 mg/kg of antibody and a weekly dose of 1,000 mg/m² of the antibody includes about 25 mg/kg of antibody. In other aspects, administering about 500 mg/m² of the antibody includes administering about 1,375 mg of antibody, administering about 750 mg/m² of the antibody includes administering about 2,065 mg of antibody, and administering about 1,000 mg/m² of the antibody includes administering about 2,750 mg of antibody. In one aspect, the 500 mg/m², 750 mg/m² and 1,000 mg/m² administration dose achieves a site occupancy of a target receptor of at least 90%.

In one aspect, the subject has a hematologic disease. In one aspect, the antibody is administered prior to, simultaneously with, or after the additional therapy. In other aspects, the antibody is administered by intravenous (IV) infusion, subcutaneous injection, or intramuscular injection. In some aspects, the amyloid deposits include aggregates of λ-light chain fibrils and/or κ-light chain fibrils. In many aspects, administering the pharmaceutical composition induces removal of amyloid deposits present in an organ or tissue. In various aspects, the organ or tissue is selected from the group consisting of heart, kidney, liver, lung, gastrointestinal tract, nervous system, muscular skeletal system, soft tissue, skin and any combination thereof.

In another embodiment, the disclosure provides a method of treating an amyloidosis disease or disorder involving a kidney, a gastrointestinal tract or a heart in a subject including administering to the subject the pharmaceutical composition described herein.

In various aspects, the method further includes administering to the subject an additional therapy. In some aspects, the additional therapy includes cyclophosphamide, bortezomib, dexamethasone, daratumumab, melphalan, lenalidomide, isatuximab, venetoclax, a stem cell transplant or a combination thereof.

In another embodiment, the disclosure provides a method of treating one or more symptoms of an amyloidosis disease or disorder involving the skin in a subject including administering to the subject the pharmaceutical composition described herein.

In one aspect, the one or more symptoms of the amyloidosis involving the skin are selected from hair loss, facial hair loss and body hair loss.

In an additional embodiment, the disclosure provides a method of inhibiting and/or reducing aggregation of light chains and amyloid fibrils in a subject including administering to the subject about 1,000 mg/m² of an antibody having a heavy chain variable domain (VH) having an amino acid sequence of SEQ ID NO:1 and a light chain variable domain (VL) having an amino acid sequence of SEQ ID NO:2.

In one embodiment, the disclosure provides a method of treating an amyloidosis disease or disorder in a subject including administering to the subject the pharmaceutical composition described herein.

In one aspect, administering the pharmaceutical composition renders the subject eligible for a stem cell transplant. In another aspect, a stem cell transplant is further performed in the subject.

In one embodiment, the invention provides a method of treating amyloidosis in a subject having multiple myeloma including administering to the subject a pharmaceutical composition including an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains, thereby treating amyloidosis in the subject.

In one aspect, the antibody binds to kappa and lambda mis-folded light chains. In another aspect, the pharmaceutical composition further includes: (a) one or more isotonic agents; (b) a buffer; and (c) a non-ionic surfactant. In some aspects, the isotonic agent is sodium acetate; the buffer is sodium chloride; and the non-ionic surfactant is polysorbate 80. In other aspects, the pharmaceutical composition includes: 30 mg/mL antibody; about 25 mM sodium acetate; about 50 mM sodium chloride; about 1% mannitol; and about 0.01%-0.05% polysorbate 80. In some aspects, the antibody administration dose is from about 500 mg/m² to 1,000 mg/m². In various aspects, the dose is selected from about 500 mg/m², about 750 mg/m² and about 1,000 mg/m². In some aspects, a weekly dose of about 500 mg/m² of antibody includes about 12.5 mg/kg of antibody, a weekly dose of about 750 mg/m² includes about 18.75 mg/kg of antibody and a weekly dose of about 1,000 mg/m² of antibody includes about 25 mg/kg of antibody. In one aspect, administering about 500 mg/m² of antibody includes administering about 1,375 mg of antibody, administering about 750 mg/m² of antibody includes administering about 2,065 mg of antibody, and administering about 1,000 mg/m² of antibody includes administering about 2,750 mg of antibody. In another aspect, the 500 mg/m², 750 mg/m² and 1,000 mg/m² administration dose achieves a site occupancy of a target of at least 90%. In one aspect, the antibody is administered weekly for at least 2, 3 or 4 weeks. In another aspect, the method further includes administering a maintenance dose of antibody to the subject thereafter. In some aspects, the maintenance dose is administered biweekly, triweekly, or monthly after the first 2, 3, 4 or more weeks. In other aspects, the pharmaceutical composition is administered by intravenous (IV) infusion, subcutaneous injection, or intramuscular injection. In one aspect, the subject is currently or has been previously treated for multiple myeloma. In some aspects, a treatment for multiple myeloma is selected from the group consisting of chemotherapy, corticosteroid, immunomodulating agent, proteasome inhibitor, histone deacetylase (HDCA) inhibitor, immunotherapy, nuclear export inhibitor, stem cell transplant, radiation therapy, surgery, and any combination thereof.

In one embodiment, the invention provides a method of treating amyloidosis in a subject having a B-cell lymphoproliferative disorder including administering to the subject a pharmaceutical composition including an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains, thereby treating the plasma cell disease in the subject.

In one aspect, the B-cell lymphoproliferative disorder is selected from the group consisting of multiple myeloma, Waldenstrom's macroglobulinemia, chronic lymphocytic leukemia, non-Hodgkin lymphoma and lymphomas associated with amyloidosis. In one aspect, the B-cell lymphoproliferative disorder is multiple myeloma. In another aspect, the subject is currently or has been previously treated for the B-cell lymphoproliferative disorder. In one aspect, a treatment for B-cell lymphoproliferative disorder includes chemotherapy.

In another embodiment, the invention provides a method of treating amyloidosis in a subject having a plasma cell disease including administering to the subject a pharmaceutical composition including an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains, thereby treating the plasma cell disease in the subject.

In one aspect, the plasma cell disease is selected from the group consisting of low-grade B-cell lymphoma, monoclonal gammopathy of undetermined significance (MGUS), and multiple myeloma. In another aspect, the antibody administration dose is from about 500 mg/m² to 1,000 mg/m². In some aspects, the dose is selected from about 500 mg/m², about 750 mg/m² and about 1,000 mg/m². In other aspects, the antibody is administered weekly for at least 2, 3 or 4 weeks. In some aspects, the method further includes administering a maintenance dose of antibody to the subject thereafter. In other aspects, the pharmaceutical composition is administered by intravenous (IV) infusion, subcutaneous injection, or intramuscular injection. In some aspects, the subject is currently or has been previously treated for the plasma cell disease. In other aspects, a treatment for plasma cell disease includes chemotherapy.

In an additional embodiment, the invention provides a method of identifying a subject having multiple myeloma as a candidate for an anti-amyloidosis treatment comprising identifying light chain (AL) amyloidosis fibrils and/or amyloid protein precursor deposition in the subject, wherein the identification of AL amyloidosis fibrils and/or amyloid protein precursor deposition in the subject is indicative of the likelihood of the subject to respond to the anti-amyloidosis treatment, and wherein the anti-amyloidosis treatment includes an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains, thereby identifying the subject as a candidate for the anti-amyloidosis treatment.

In one aspect, the subject is currently or has been previously treated for multiple myeloma. In another aspect, the method further includes administering to the subject a treatment for multiple myeloma. In some aspects, a treatment for multiple myeloma is selected from the group consisting of chemotherapy, corticosteroid, immunomodulating agent, proteasome inhibitor, histone deacetylase (HDCA) inhibitor, immunotherapy, nuclear export inhibitor, stem cell transplant, radiation therapy, surgery, and any combination thereof.

### BRIEF DESCRIPTION OF THE DRAWINGS

FIGS. 1A-1C illustrate the characterization of the antibody charge heterogeneity assessed by three independent methods. FIG. 1A shows the antibody charge heterogeneity characterization by capillary zone electrophoresis (CZE) separation. FIG. 1B shows the antibody charge heterogeneity characterization by capillary isoelectric focusing (cIEF) separation. FIG. 1C shows the antibody charge heterogeneity characterization by cation exchange chromatography (CEX).

FIGS. 2A-2B illustrate the comparison of individual antibody concentrations in a phase 2 study as compared to the phase 1b study. FIG. 2A is a line graph showing antibody concentrations in patients. FIG. 2B is a line graph showing antibody concentrations in patients on a semi-log scale.

FIGS. 3A-3B illustrate the comparison of individual antibody mean concentrations in a phase 2 study as compared to the phase 1b study. FIG. 3A is a line graph showing antibody mean concentrations in patients on a linear scale. FIG. 3B is a line graph showing antibody mean concentrations in patients on a semi-log scale.

FIG. 4 is a line graph illustrating the antibody mean concentrations from the Phase 1b study, and the determination of the Cₘᵢₙ.

FIG. 5 is a graph bar illustrating the dose proportionality assessment for Cₘₐₓ/dose.

FIG. 6 is a graph bar illustrating the dose proportionality assessment for Cₘᵢₙ/dose.

FIG. 7 is a graph bar illustrating the dose proportionality assessment for AUCτ/dose.

FIGS. 8A-8B show the sequences of the heavy and light chains of the antibody, including the CDR regions. FIG. 8A shows the sequence of the heavy chain (SEQ ID NO:1). FIG. 8B shows the sequence of the light chain, including the CDR regions (SEQ ID NO:2).

FIG. 9 is a graph illustrating Global Longitudinal Strain (GLS) % change from baseline to 12 weeks.

### DETAILED DESCRIPTION

This disclosure relates to the discovery that the antibodies described herein, alone or in combination with an additional therapy, are effective for the treatment of amyloidosis diseases and disorders including amyloidosis deposit in patients having a plasma cell disease or disorder. In some aspects, the amyloidosis diseases can be relapsed or refractory.

Before the present compositions and methods are described, it is to be understood that this disclosure is not limited to particular compositions, methods, and experimental conditions described, as such compositions, methods, and conditions may vary. It is also to be understood that the terminology used herein is for purposes of describing particular embodiments only, and is not intended to be limiting, since the scope of the present disclosure will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein, which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, it will be understood that modifications and variations are encompassed within the spirit and scope of the instant disclosure. The preferred methods and materials are now described.

In one embodiment, the disclosure provides a composition including an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains, one or more isotonic agents, a buffer, and a non-ionic surfactant.

The antibody of the present disclosure is used for treating amyloidosis diseases and disorders. An antibody consists of four polypeptides: two identical copies of a heavy (H) chain polypeptide and two copies of a light (L) chain polypeptide. There are 5 types of heavy chains: IgG, IgM, IgA, IgD, or IgE; and two possible light chains: kappa (κ) or lambda (λ). Each heavy chain contains one N-terminal variable (VH) region and three C-terminal constant (CH1, CH2 and CH3) regions, and each light chain contains one N-terminal variable (V_{L} or V_{K}, or V_{λ} or V_{κ}) region and one C-terminal constant (CL) region. Each variable domain of the light and heavy chain in an antibody also includes three segments called complementarity-determining regions (“CDR”) or hypervariable regions. Each CDR in a light chain, together with the corresponding CDR in the adjacent heavy chain, form an antigen-binding site of the antibody. The variable regions of each pair of light and heavy chains form the antigen binding site of an antibody, whereas the constant region provides structural support and modulates the immune response initiated by the antigen binding.

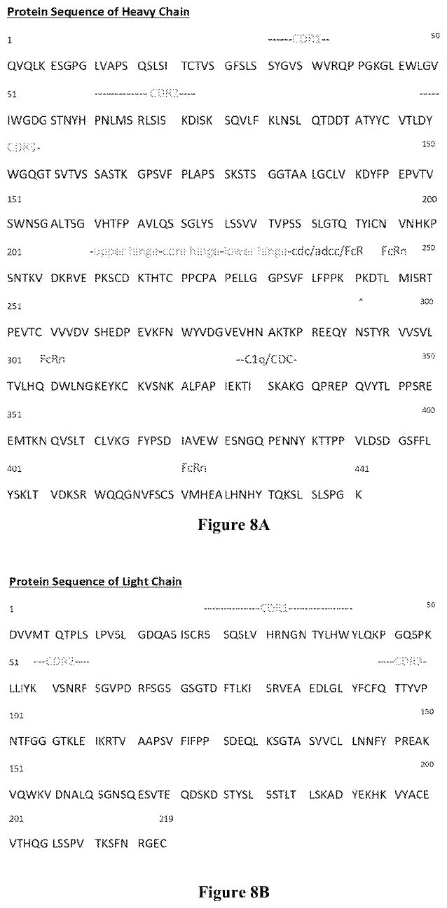

The antibody described herein has a V_{K} region (SEQ ID NO: 2) and a V_{H} region (SEQ ID NO: 1) as shown in Table 1 below and in FIG. 8. The CDR sequences for the heavy and light chains and provided in Table 2 and FIG. 8.

TABLE 1
Monoclonal antibody variable sequences

| | |
|---|---|
| SEQ ID | V_{K} region: |
| NO: 2 | DVVMTQTPLSLPVSLGDQASISCRSSQSLVHRNGNTYLHWYLQ |
| | KPGQSPKLLIYKVSNRFSGVPDRFSGSGSGTDFTLKISRVEAE |
| | DLGLYFCFQTTYVPNTFGGGTKLEIK |
| SEQ ID | V_{H} region: |
| NO: 1 | QVQLKESGPGLVAPSQSLSITCTVSGFSLSSYGVSWVRQPPGK |
| | GLEWLGVIWGDGSTNYHPNLMSRLSISKDISKSQVLFKLNSLQ |
| | TDDTATYYCVTLDYWGQGTSVTVSS |

TABLE 2
Monoclonal antibody CDR sequences

| | |
|---|---|
| SEQ ID NO: 3 | CDRL1 |
| | SSQSLVHRNGNTYLHWY |
| SEO ID NO: 4 | CDRL2 |
| | KVSNRF |
| SEO ID NO: 5 | CDRL3 |
| | QTTYVP |
| SEQ ID NO: 6 | CDRHI |
| | SYGVSWV |
| SEQ ID NO: 7 | CDRH2 |
| | PNLMSRLSISKD |
| SEO ID NO: 8 | CDRH3 |
| | DYWGQG |

The genes encoding the V_{H} and V_{K} regions can be cloned to produce a chimeric antibody using known human antibody C_{H} and C_{K} sequences. It is believed that the antibody of the disclosure binds to an epitope expressed by the β-pleated sheet configuration of amyloids, but also to AL amyloid fibrils.

The disclosed antibodies can include any types of human constant regions and/or framework regions. For example, the disclosed humanized and chimeric antibodies can include the constant regions and/or framework regions of a human IgG (including IgG1, IgG2, IgG3, and IgG4), IgA, IgE, IgF, IgH, or IgM. In one aspect, the disclosed antibody includes a human IgG1 constant region.

Antibodies can be cleaved with the proteolytic enzyme papain, which causes each of the heavy chains to break, producing three separate antibody fragments. The two identical units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding but is important in later processes involved in elimination of the antigen from the body. “Fv” is the minimum antibody fragment that contains a complete antigen-recognition and -binding site. This region consists of a dimer of one heavy- and one light-chain variable domain in tight, noncovalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. “Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

In some aspects, the disclosed antibodies include one or more substitutions, insertions, or deletions, so long as the antibody maintains the ability to bind to amyloid fibrils (e.g., kappa and/or lambda light chain fibrils). For example, in some aspects, the antibody of the present disclosure includes heavy and light chains with about 85%, about 86%, about 87%, about 88%, about 89%, about 90%, about 91%, about 92%, about 93%, about 94%, about 95%, about 96%, about 97%, about 98%, about 99%, or about 100% identity compared to the corresponding heavy and light chain sequences disclosed herein, so long as the antibody maintains the ability to bind to amyloid fibrils. In other aspects, the antibody of the present disclosure includes CDRs that have about 85%, about 86%, about 87%, about 88%, about 89%, about 90%, about 91%, about 92%, about 93%, about 94%, about 95%, about 96%, about 97%, about 98%, about 99%, or about 100% identity compared to the corresponding CDR sequences disclosed herein, so long as the antibody maintains the ability to bind to amyloid fibrils.

The presently disclosed antibodies can bind and neutralize toxic circulating amyloid proteins that have not yet formed deposits or fibrils and can dissolve amyloid deposits. Indeed, corresponding murine and chimeric antibodies were demonstrated to bind to fibrils and to dissolve human amyloidomas in mice.

An antibody useful in the compositions and methods of the disclosure may be a monoclonal antibody including CDR sequences of SEQ ID NOs: 3-8. These antibodies bind to an epitope presented by the β-pleated sheet configuration of amyloid fibrils. In one aspect, the antibody of the present disclosure binds to kappa and lambda mis-folded light chains. As used herein “binding to mis-folded light chains” refers to the binding specificity of the antibody that recognizes and binds to abnormal light chains (kappa and lambda), but does not recognize and does not bind to non-aggregated or free light chains that are properly folded (in a native and typical conformation). Native light chain or fragments thereof are functional peptide, that are normally degraded through proteolysis. Upon mis-folding, a peptide can loss its physiological structure and function; the conformational change renders the peptide non-functional, and more stable, preventing its degradation through proteolysis. Accumulated mis-folded light chains can aggregate with one another to form amyloid fibrils, then can further aggregates with one another or with additional mis-folded light chains. Amyloids fibrils are fibrous deposits that cannot be degraded by the cells, and that accumulate in plaques around cells disrupt the healthy function of tissues and organs. Amyloid deposits typically comprise aggregated mis-folded kappa light chain, or mis-folded lambda light chain in a given patient; and usually do not include both kappa and lambda light chains in an aggregate. The antibody of the present disclosure recognizes both kappa and lambda light chains in their mis-folded conformation and does not recognize kappa or lambda chain in their physiological conformation (properly folded light chains). An aggregate is not required to include both kappa and lambda mis-folded light chains to be recognized by the antibody.

The composition can include one or more isotonic agents, for example, the composition can include 1, 2, 3, 4 or more isotonic agents. In some aspects, the one or more isotonic agents are selected from sugars, poly-alcohols such as mannitol, sorbitol, or sodium chloride.

The composition can include a buffer, to keep the pH of the composition at a nearly constant value in a wide variety of chemical applications. In some aspects, the buffer is sodium acetate.

The composition can include a non-ionic surfactant, to lower surface tension or interfacial tension. For example, the composition can include a non-ionic surfactant selected from: ethoxylate, fatty alcohol ethoxylates (such as narrow-range ethoxylate, octaethylene glycol monododecyl ether, and pentaethylene glycol monododecyl ether), alkylphenol ethoxylates (APEs or APEOs, such as nonoxynols and Triton X-100), fatty acid ethoxylates, special ethoxylated fatty esters and oils, ethoxylated amines and/or fatty acid amides (such as polyethoxylated tallow amine, cocamide monoethanolamine, and cocamide diethanolamine), terminally blocked ethoxylates (such as poloxamers), fatty acid esters of polyhydroxy compounds, fatty acid esters of glycerol (such as glycerol monostearate and glycerol monolaurate), fatty acid esters of sorbitol (such as spans: sorbitan monolaurate, sorbitan monostearate, and sorbitan tristearate; and Tweens, or polysorbates: Tween 20, Tween 40, Tween 60, and Tween 80), fatty acid esters of sucrose, and alkyl polyglucosides (such as cecyl glucoside, lauryl glucoside and octyl glucoside).

In one aspect, the composition includes the antibody described herein, sodium acetate, sodium chloride, mannitol and polysorbate 80.

In one aspect, the composition includes from about 20 to 40 mg/mL of antibody. In another aspect, the composition includes from about 15 to 35 mM sodium acetate. In yet another aspect, the composition includes from about 25 to 75 mM sodium chloride. In one aspect, the composition includes from about 0.5 to 5% mannitol. In another aspect, the composition includes from about 0.001 to about 0.1% polysorbate 80. In yet another aspect, the composition has a pH from about 5 to 6.

In one embodiment, the composition includes about 30 mg/mL of antibody; about 25 mM sodium acetate; about 50 mM sodium chloride; about 1% mannitol; about 0.01-0.05% polysorbate 80; and a pH of about 5.5.

In one aspect, the composition includes 30 mg/ml of antibody, about 25 mM sodium acetate, about 50 mM sodium chloride, about 1% mannitol, about 0.01-0.05% polysorbate 80, and has a pH of about 5.5 in a vial or ampule, for example.

In another aspect, the antibody is a mixture of antibody molecules including a native fraction, a reduced fraction, and a glycosylated or deglycosylated fraction having a heterogeneous charge. The mixture of antibody molecules can include those with a native structure (defining a native fraction), a reduced structure (defining a reduced fraction), and a glycosylated or deglycosylated structure (defining a variably glycosylated or deglycosylated fraction), any of which have a heterogeneous charge.

Post-translational modifications (PTMs) induced by chemical and enzymatical intra- and extracellular mechanisms can affect the micro-heterogeneity, and charge heterogeneity of recombinant antibodies and thereby influence important quality attributes, such as stability, solubility, efficacy, safety, pharmacodynamics and pharmacokinetics. The recombinant cell line, the culture media and the process settings may also affect these quality attributes. The distribution of surface charge variants is also an important measure of antibodies' heterogeneity.

Charge variations of proteins differ within the type of modification; some PTMs directly modify the net charge of proteins while others induce conformational changes and variation of local charge distribution. Charge species with a lower isoelectric point (pI) than the main fraction of the product are defined as acidic variants and generated by sialylation, deamidation of asparagine and glutamine, glycation and other mechanisms. Glycation, for instance, is a non-enzymatic reaction where a reducing sugar molecule, most commonly glucose, is covalently bound to a reactive amino group. Basic variants are defined as species with a higher pI than the main fraction and generated by incomplete C-terminal lysine clipping of the heavy chains, as well as by fragmentation and aggregation. Cyclization of N-terminal glutamines to form pyroglutamic acids is another example of positive charge loss of antibodies by the conversion of the N-terminal amine to a neutral amide. Deamidation is a common degradation pathway of proteins that modifies non-enzymatically asparagine residues to aspartic acid and/or isoaspartic residues and/or succinimide intermediates, resulting in the appearance of a negative charge. Some other PTMs affect the local charge distribution without modification of the net charge of the proteins, such as methionine oxidation or aspartic acid isomerization leading to the insertion of an extra methyl group into the backbone protein to form isoaspartic acid. The modifications of charge profiles can potentially affect the structure and the biological activity of proteins. Other PTMs that may impact the functionality of the antibody of the disclosure include fuculose and mannose, which can respectively generate fucosylated and mannosylated antibodies or fragments thereof.

The composition of the present disclosure includes an antibody that can be present in several forms, each form defining a fraction of the antibody composition. For example, the antibody can be present in a native form, which is the main form of the antibody in the absence of any stress, and which represents the main fraction. The antibody can also be present in a reduced form, or in a reduced and deglycosylated form, which represent the reduced fraction and the reduced and deglycosylated fraction, respectively.

In one aspect, the native fraction includes sialylated species, neutral species, and/or galactosylated, fucosylated and/or mannosylated neutral species. Other glycosylated forms might include fucosylated and non-fucosylated forms, and high mannose forms. Since intact antibodies are heterodimeric and contain two heavy chain molecules, the glycosylation on each chain in an intact antibody may be the same or different from that of the other heavy chain. In another aspect, the reduced fraction includes light chains with glycated lysines. As used herein, the phrase “light chain with glycated lysines” is meant to include various levels of glycation of the lysines. For example, no lysine, one lysine, some lysines, or all the lysines of the light chain can be glycated.

The analysis of the surface charge distribution of monoclonal antibodies provides aggregated information about these modifications. Common analytical methods for the determination of charge heterogeneities of antibodies include capillary isoelectric focusing (cIEF) and ion exchange chromatography (IEX). Both methods are widely used, but IEX methods, using a salt gradient elution, are recognized as the standard and are routinely use. The major limitation of IEX is the salt buffer system that need to be adapted for every antibody. However, the use of pH gradients was shown to be product-independent and a cation exchange chromatography (CEX) method with a linear pH gradient for the determination of charge heterogeneity of antibody can also be used. These studies report the impact of forced stress degradation at elevated temperature or alkaline pH on mAbs charge variants. Degradations observed with such stresses mainly lead to an increase of acidic species, reflecting deamidation or oxidation reactions of proteins. the charge heterogeneity can also be measured by capillary zone electrophoresis (CZE) separation.

In one aspect, the antibody is a mixture including intact antibodies, halfmer fragments, incomplete antibody fragments, other fragments and/or aggregates thereof. In some aspects, the halfmer is an antibody molecule that includes one or two heavy chains (HC) and one light chain (LC). In other aspects, the incomplete antibody is an antibody missing a C-terminal region of a HC. In some aspects, the other fragment includes HC retaining C-terminal lysine. In various aspects, antibody aggregates, or antibody fragments may or may not retain C-terminal lysine.

The composition may be formulated for intravenous, subcutaneous, intraperitoneal, intramuscular, oral, nasal, pulmonary, ocular, vaginal, or rectal administration. In some embodiments, the antibodies are formulated for intravenous, subcutaneous, intraperitoneal, or intramuscular administration, such as in a solution, suspension, emulsion, liposome formulation, etc.

Pharmacologically acceptable carriers for various dosage forms are known in the art. For example, excipients, lubricants, binders, and disintegrants for solid preparations are known; solvents, solubilizing agents, suspending agents, isotonicity agents, buffers, and soothing agents for liquid preparations are known. In some embodiments, the pharmaceutical compositions include one or more additional components, such as one or more preservatives, antioxidants, stabilizing agents and the like.

Additionally, the disclosed pharmaceutical compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

In the present disclosure, an antibody is administered to a subject (e.g., a human patient) suffering from an amyloidosis disease or disorder to promote the degradation and removal of at least some of the amyloid fibrils that have become deposited in the organs of the patient and/or that are circulating in the patient's bloodstream. In various embodiments, the present disclosure provides methods of treatment including the administration of the antibody described herein. In some aspects, a therapeutically effective amount of the antibody is administered. A typical route of administration is parenterally (e.g., intravenously, subcutaneously, or intramuscularly), as is well understood by those skilled in the medical arts. Other routes of administration are, of course, possible. Administration may be by single or multiple doses, alone or in combination with an additional therapy, as discussed below. The amount of antibody administered, and the frequency of dosing may be optimized by the physician for the particular patient.

In one embodiment, the disclosure provides a method of decreasing an amount of amyloid deposits in a subject including administering to the subject about 1,000 mg/m² of an antibody having a heavy chain variable domain (VH) having an amino acid sequence of SEQ ID NO:1 and a light chain variable domain (VL) having an amino acid sequence of SEQ ID NO:2.

There are many different types of amyloidosis diseases and disorders, including hereditary and sporadic forms, that are caused by outside factors, such as inflammatory diseases or long-term dialysis. Amyloidosis can affect different organs in different people, and there are different types of amyloid. Amyloidosis frequently affects the heart, kidneys, liver, spleen, nervous system and digestive tract. Severe amyloidosis can lead to life-threatening organ failure. Many types affect multiple organs, while others affect only one part of the body. Signs and symptoms of amyloidosis may include, but are not limited to: swelling of the ankles and legs; severe fatigue and weakness; shortness of breath; numbness, tingling or pain in the hands or feet, especially pain in the wrist (carpal tunnel syndrome); diarrhea, possibly with blood, or constipation; unintentional, significant weight loss; an enlarged tongue; skin changes, such as thickening or easy bruising, and purplish patches around the eyes; an irregular heartbeat; or difficulty swallowing.

In general, amyloidosis is caused by the buildup and aggregation of mis-folded light chain proteins of fragment thereof. Amyloid can be produced and deposited in any tissue or organ. The specific cause of the condition, and the affected organs depend on the type of amyloidosis. There are several types of amyloidosis or amyloid diseases, including AL amyloidosis, AA amyloidosis, hereditary amyloidosis, wild-type amyloidosis, and localized amyloidosis.

AL amyloidosis (immunoglobulin light chain amyloidosis), or primary amyloidosis is the most common type and can affect the heart, kidneys, skin, nerves and liver. AL amyloidosis occurs when the bone marrow produces abnormal antibodies that cannot be broken down. The antibodies are deposited in various tissues as amyloid plaques, which interfere with normal function of the tissue or organ.

AA amyloidosis, or secondary amyloidosis generally affects the kidneys but occasionally also affects the digestive tract, liver, spleen or heart. It often occurs along with chronic infectious or inflammatory diseases, such as rheumatoid arthritis or inflammatory bowel disease. Improved treatments for severe inflammatory conditions have resulted in a sharp decline in the number of cases of AA amyloidosis in developed countries.

Hereditary amyloidosis (familial amyloidosis) is an inherited disorder that usually affects the liver, nerves, heart, and/or kidneys. Many different types of gene abnormalities present at birth are associated with an increased risk of amyloid disease or hereditary amyloidosis. The type and location of an amyloid gene abnormality can affect the risk of certain complications, the age at which symptoms first appear, and the way the disease progresses over time. It most commonly happens when the gene encoding a liver transthyretin (TTR) protein is mutated.

Wild-type amyloidosis is an amyloidosis subtype that occurs when the TTR protein made by the liver is normal but produces amyloid for unknown reasons. Formerly known as senile systemic amyloidosis, wild-type amyloidosis tends to affect men over age 70 and typically targets the heart. It can also cause carpal tunnel syndrome.

Localized amyloidosis. This type of amyloidosis often has a better prognosis than the subtypes that affect multiple organ systems. Typical sites for localized amyloidosis include the bladder, skin, throat or lungs.

In various aspect, the antibody described herein can be used for the treatment of any misfolding protein disorders, including AL, AA, TTR, wild-type and localized amyloidosis. In one aspect, the amyloidosis is selected from the group consisting of light chain (AL) amyloidosis, autoimmune (AA) amyloidosis and hereditary (TTR) amyloidosis.

The pharmaceutical composition of the present disclosure can be used for the treatment any type of amyloidosis. As used herein, the phrase “decreasing an amount of amyloid deposit” is meant to refer to either the reduction of the amount of deposit, the reduction of the size of the deposit, the active deconstruction of the amyloid fibrils, the inhibition of the aggregation of light chains with amyloid fibrils, and/or the inhibition of the formation of new amyloid deposit in a subject.

Decreasing an amount of amyloid deposit generally relies on the administration of a “therapeutically effective amount” of the antibody described herein, referring to an antibody dose or plasma concentration in a subject, respectively, that provides the specific pharmacological effect for which the antibody is administered in a subject in need of such treatment, i.e., to reduce, ameliorate, or eliminate the amount, size, and aggregation capability of the amyloid fibrils. It is emphasized that a therapeutically effective amount or therapeutic level of a drug will not always be effective in treating the conditions/diseases described herein, even though such dosage is deemed to be a therapeutically effective amount by those of skill in the art. The therapeutically effective amount may vary based on the route of administration and dosage form, the age and weight of the subject, and/or the subject's condition, including the type and stage of the amyloidosis at the time that treatment commences, among other factors.

A therapeutically effective amount may be the dose or amount sufficient to induce a “therapeutic response” in a subject, such as an improvement in at least one measure of amyloid disease, such as a reduction in the size of existing amyloid deposits or plaques, a decrease in the rate of amyloid deposition, or improved organ function as measured by standard techniques. For instance, in patients with amyloid deposits in the heart, improved organ function (i.e., a therapeutic response) may be indicated by a decrease in the level of the patient's N-terminal pro b-type natriuretic peptide (NT-proBNP) or a decrease in the patient's New York Heart Association (NYHA) Functional Classification level. Heart function improvement may also be evaluated by measuring cardiac troponin levels, by analyzing cardiac MRI and echocardiogram. In patients with amyloid deposits in the kidneys, improved organ function (i.e., a therapeutic response) may be indicated by a decrease in proteinuria or the rate of protein output in the urine and estimated glomerular filtration rate (eGFR). In patients with amyloid deposits in the liver, organ improvement may be indicated by a decrease in the abdominal distention, hepatomegaly, ascites, and/or oliguria. Liver improvement may be detected by improved alkaline phosphatase (ALP) levels and/or serum y-glutamyltransferase (GGT) levels. Improvement of other metrics, such as hyperlipidemia, coagulation abnormalities, thrombocytopenia, prothrombin time (PT), erythrocyte sedimentation rate, alanine aminotransferase (ALT) and/or aspartate aminotransferase (AST), serum albumin and complement fragment levels can also indicate improvement of liver function, when those parameters were evaluated prior to any treatment, as they lack specificity for hepatic amyloidosis. In patients with amyloid deposits in the gastrointestinal (GI) tract, organ improvement may be indicated by a reduction of the symptoms caused by the deposits, such as altered motility, gastrointestinal bleeding, malabsorption, weight loss, anorexia, vomiting, nausea, hematomas, erosions and ulcerations, or a nodular gastritis. Such improvement may be evaluated using conventional imaging (e.g., echography, computed tomography scanner, X-ray, endoscopy and the like).

Amyloid deposits can also occur in or at proximity to nerves, which may lead to amyloid neuropathies such as sensorimotor polyneuropathy, characterized by symptoms of neuropathic pain, numbness, and in advanced cases weakness. Such symptoms begin in the feet and ultimately progress to the proximal legs and hands (including lateral palm and fingers). In patients with amyloid neuropathies, improvement may be evaluated by electrophysiologic tests such nerve conduction studies (NCS), electromyography (EMG), autonomic function testing (AFT), and quantitative sudomotor axon reflex testing (QSART).

As used herein, the terms “individual”, “patient”, or “subject” can be used interchangeably, and refer to an individual organism, a vertebrate, a mammal (e.g., a bovine, a canine, a feline, or an equine), or a human that is being administered the antibody of the present disclosure. In a preferred embodiment, the individual, patient, or subject is a human.

The antibody of the present disclosure can be administered to any subject having amyloidosis, independently of the treatment previously received, if any, prior to the administration of the presently described antibody. The antibody can be administered whether the amyloidosis disease or disorder has been previously treated or has never been treated.

In one aspect, the subject is newly diagnosed with an amyloidosis disease or disorder prior to administration of the antibody. In other aspects, the subject has been previously treated for amyloidosis disease or disorder prior to administration of the antibody.

In one embodiment, the disclosure provides a method of treating an amyloidosis disease or disorder in a subject including administering to the subject the pharmaceutical composition of the disclosure and an additional therapy.

The terms “treatment” or “treating” refer to reducing, ameliorating or eliminating one or more symptoms or effects of the amyloidosis, including but not limited to clearance or degradation of amyloid plaques or deposits, improving organ function of organs effected by the disease (e.g., the heart, kidney, liver, etc.), and increasing the patient's lifespan or 5-year survival. As used herein the phrase “treating amyloidosis” is meant to include the treatment of any disease or disorder that is characterized by the accumulation of amyloid deposit in a tissue or organ. Such treatment may be efficient for the removal of existing amyloid deposits, facilitating clearing of amyloid deposits from organs and tissues, the inhibition of the aggregation of amyloid fibrils with mis-folded lights chains, the prevention of amyloid fibrils aggregation, and the prevention of future amyloid deposition in organs and tissues.

The terms “administration of” and or “administering” should be understood to mean providing the antibody of the disclosure in a therapeutically effective amount to the subject in need of treatment. Administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization.

In one aspect, the antibody is administered by intravenous (IV) infusion, subcutaneous injection or intramuscular injection.

In some aspects, administration can be in combination with one or more additional therapy. The phrases “combination therapy”, “combined with” and the like refer to the use of more than one medication or treatment simultaneously to increase the response. The pharmaceutical composition of the present disclosure might for example be used in combination with other drugs or treatment in use to treat amyloidosis. Specifically, the administration of the pharmaceutical composition containing the antibody of the disclosure to a subject can be in combination with a plasma cell directed therapy such as cyclophosphamide, bortezomib, dexamethasone, daratumumab, melphalan, lenalidomide, isatuximab, venetoclax, a stem cell transplant, or a combination thereof. Such therapies can be administered prior to, simultaneously with, or following administration of an antibody or antibody composition of the present disclosure.

Cyclophosphamide is a chemotherapeutic agent that suppress the immune system. Cyclophosphamide can induce the formation of DNA crosslinks both between and within DNA strands at guanine N-7 positions in cells that have low levels of ALDH. DNA crosslinked are irreversible and lead to cell apoptosis. Cyclophosphamide induces beneficial immunomodulatory effects in adaptive immunotherapy, notably by eliminating T regulatory cells (CD4+CD25+ T cells).

Bortezomib is an anti-cancer medication that binds the catalytic site of the 26S proteasome with high affinity and specificity. By inhibiting the proteasome, bortezomib prevents degradation of pro-apoptotic factors, thereby triggering programmed cell death in neoplastic cells.

Dexamethasone is a corticosteroid medication used in the treatment of many conditions, including rheumatic problems, a number of skin diseases, severe allergies, asthma, chronic obstructive lung disease, croup, brain swelling, eye pain following eye surgery, and along with antibiotics in tuberculosis.

CyBorD is a combination of cyclophosphamide, bortezomib and dexamethasone that is usually used in the treatment of multiple myeloma.

Daratumumab is an IgG1k monoclonal antibody directed against CD38, which is overexpressed in multiple myeloma cells. Daratumumab binds to CD38, and induced apoptosis via antibody-dependent cellular cytotoxicity, complement-dependent cytotoxicity or antibody-dependent cellular phagocytosis.

Melphalan is a chemotherapeutic agent used to treat multiple myeloma, ovarian cancer, melanoma, and amyloidosis. It is orally or intravenously administered, and chemically alters DNA nucleotide guanine through alkylation. Alkylation causes linkages between strands of DNA that in turn inhibits DNA synthesis and RNA synthesis, and cause cytotoxicity in both dividing and non-dividing tumor cells. Common side effect of melphalan includes bone marrow suppression, which is beneficial for the treatment of amyloidosis.

Lenalidomide is used to treat multiple myeloma (MM) and myelodysplastic syndromes (MDS) and can be administered at least with one other treatment and generally together with dexamethasone.

Isatuximab is a monoclonal antibody used for the treatment of multiple myeloma. Isatuximab selectively binds to CD38 expressed at the surface of hematopoietic and multiple myeloma cells, which induces apoptosis of tumor cells and activates immune effector mechanisms such as complement dependent cytotoxicity (CDC), antibody-dependent cellular phagocytosis (ADCP), and antibody-dependent cell-mediated cytotoxicity (ADCC).

Venetoclax is a BH3-mimetic that blocks the anti-apoptotic B-cell lymphoma-2 (Bcl-2) protein, leading to programmed cell death of CLL cells.

As used herein, the phrase “plasma cell directed therapy” is meant to refer to any directed or targeted therapy that can be used to specifically inhibit plasma cells (plasma B cell or antibody-producing cells). Plasma cell-targeted therapies include, but are not limited to lenalidomide, bortezomib, dexamethasone, proteasome inhibitor and combination thereof.

In one aspect, the antibody is administered prior to, simultaneously with, or after the additional therapy. In other aspects, the antibody is administered prior to the additional therapy.

In one embodiment, the invention provides a method of treating amyloidosis in a subject having a B-cell lymphoproliferative disorder including administering to the subject a pharmaceutical composition including an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains, thereby treating the plasma cell disease in the subject.

B-cell lymphoproliferative disorders can be associated with systemic AL amyloidosis. AL amyloidosis associated with lymphoproliferative disorders appears to be caused by monoclonal immunoglobulin light chains produced by the neoplastic B-cells that arise in these disorders. Patients with systemic amyloidosis have high levels of M-protein, multiorgan involvement with frequent cardiac involvement, and nephrotic syndrome. Among the lymphoproliferative disorders that have been described that cause Ig monoclonal gammopathies are Waldenstrom's macroglobulinemia (also named lymphoplasmacytic lymphoma), chronic lymphocytic leukemia, and other lymphomas associated with amyloidosis (types of non-Hodgkin lymphoma).

Multiple myeloma is a clonal malignancy of terminally differentiated B lymphocytes characterized by the expansion of clonal plasma cells in the bone marrow resulting in suppression of normal hematopoiesis, production of monoclonal immunoglobulins or fragments (light or heavy chain), immunosuppression, nephropathy and neuropathy. These findings often result from direct injury or accumulation of immunoglobulins (heavy or light chain) in various organs. The toxic effects and organ dysfunction caused by immunoglobulin deposition, however, differ in severity, clinical presentation and prognosis from that caused by ‘amyloidogenic’ light chain deposition as seen in AL amyloidosis.

The presence of systemic amyloidosis as a co-morbid condition to myeloma and lymphoma and organ involvement is associated with inferior outcomes.

In one aspect, the B-cell lymphoproliferative disorder is selected from the group consisting of multiple myeloma, Waldenstrom's macroglobulinemia, chronic lymphocytic leukemia, non-Hodgkin lymphoma and lymphomas associated with amyloidosis.

In various aspects, the B-cell lymphoproliferative disorder is multiple myeloma.

In another aspect, the subject is currently or has been previously treated for the B-cell lymphoproliferative disorder. In one aspect, a treatment for B-cell lymphoproliferative disorder includes chemotherapy.

In one embodiment, the invention provides a method of treating amyloidosis in a subject having multiple myeloma including administering to the subject a pharmaceutical composition including an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains, thereby treating amyloidosis in the subject.

Multiple myeloma (MM), also known as plasma cell myeloma and simply myeloma, is a cancer of plasma cells, a type of white blood cell that normally produces antibodies. MM is often initially asymptomatic, with bone pain, anemia, kidney dysfunction, and infections occurring as the disease progresses. There is no known cause for MM, but obesity, radiation exposure, family history, and certain chemicals are considered risk factors.

B lymphocytes are produced in the bone marrow and relocate to the lymph nodes upon maturation. As they progress, they mature and display different proteins on their cell surfaces. When they are activated to secrete antibodies, they are known as plasma cells. Multiple myeloma develops in B lymphocytes after they have left the germinal center of lymph node. The normal cell line most closely associated with MM cells is generally taken to be either an activated memory B cell or a precursor to plasma cells, the plasmablast. The immune system keeps the proliferation of B cells and the secretion of antibodies under tight control. Genetic events, such as mutations or translocation can be responsible for important modulation of B cell proliferation that can lead to the development of MM.

Multiple myeloma develops from monoclonal gammopathy of undetermined significance that progresses to smoldering myeloma. Abnormal plasma cells produce abnormal antibodies and/or monoclonal free light chains, which can cause kidney problems and overly thick blood. The plasma cells can also form a mass in the bone marrow or soft tissue. When one tumor is present, it is called a plasmacytoma; the presence of more than one tumor lead to the appellation multiple myeloma. Multiple myeloma is diagnosed based on blood or urine tests finding abnormal antibodies, bone marrow biopsy finding cancerous plasma cells, and medical imaging finding bone lesions. Another common finding is high blood calcium levels. Because many organs can be affected by myeloma, the symptoms and signs vary greatly. Fatigue and bone pain are the most common symptoms at presentation. Because of the various effects induced by MM, there are various ways to diagnose the disease. MM can be diagnosed through blood tests, histopathology, medical imaging, or the use of the diagnostic criteria.

Blood test usually rely on the detection of the presence of a paraprotein (monoclonal protein, or M protein and/or monoclonal free light chains); increased levels of all classes of immunoglobulin, especially IgG paraproteins, IgA and IgM; increased levels of isolated light and or heavy chains (κ- or λ-light chains or any of the five types of heavy chains α-, γ-, δ-, ε- or μ-heavy chains); raised calcium level (when osteoclasts are breaking down bone, releasing it into the bloodstream), and/or raised serum creatinine level due to reduced kidney function.

Histopathology can be used to estimate the percentage of bone marrow occupied by plasma cells, by performing a bone marrow biopsy. Characterization of the particular cell types based on the expression of surface proteins can be used to detect plasma cells that express immunoglobulin in the cytoplasm and occasionally on the cell surface. Myeloma cells are often CD56, CD38, CD138, and CD319 positive and CD19, CD20, and CD45 negative. The morphology of the cells can also be studies and used as a distinctive characteristic of myeloma cells.

The diagnostic examination of a person with suspected multiple myeloma typically includes a skeletal survey or PET-CT. If skeletal survey or PET-CT is negative a whole-body MRI is performed to detect bone lesions.

Diagnostic criteria have been developed to help the diagnosis of MM. A diagnostic of symptomatic myeloma is asserted when a patients meets at least one of the following criteria: clonal plasma cells account for >10% on a bone marrow biopsy or (in any quantity) in a biopsy from other tissues (plasmacytoma); a monoclonal protein (myeloma protein) is detected in either the serum or urine and it is higher than 3 g/dL (except in cases of true non-secretory myeloma); and evidence of end-organ damage related to the plasma cell disorder (related organ or tissue impairment, CRAB) are found.

The CRAB criteria encompass the most common signs of multiple myeloma:
Calcium: serum calcium >0.25 mmol/l (>1 mg/dl) higher than the upper limit of normal or >2.75 mmol/l (>11 mg/dl);
Renal insufficiency: creatinine clearance <40 ml per minute or serum creatinine >1.77 mol/1 (>2 mg/dl);
Anemia: hemoglobin value of >2 g/dl below the lowest limit of normal, or a hemoglobin value <10 g/dl;
Bone lesions: one or more osteolytic lesion on skeletal radiography, CT, PET/CT or MRI.

In multiple myeloma, staging helps with prognostication but does not guide treatment decisions. MM can be classified as stage I-III. Stage I: β2 microglobulin (β2M)<3.5 mg/L, albumin≥3.5 g/dL, normal cytogenetics, no elevated LDH. Stage II: Not classified under Stage I or Stage III. Stage III: β2M≥5.5 mg/L and either elevated LDH or high-risk cytogenetics [t(4,14), t(14,16), and/or del(17p)].

A myeloma protein is an abnormal antibody (immunoglobulin) or (more often) a fragment thereof, such as an immunoglobulin light chain, that is produced in excess by an abnormal monoclonal proliferating plasma cell. Other terms for such a protein include M protein, M-component, M-spike, spike protein, monoclonal protein or paraprotein. This proliferation of the myeloma protein has several deleterious effects on the body, including impaired immune function, abnormally high blood viscosity (“thickness” of the blood), and kidney damage.

Myeloma is a malignancy of plasma cells. Plasma cells produce immunoglobulins, each consisting of pairs of heavy and light chains. In multiple myeloma, a malignant clone, a rogue plasma cell, reproduces in an uncontrolled fashion, resulting in overproduction of the specific antibody the original cell was generated to produce, resulting in a “spike” on the normal distribution, which is called an M spike (or monoclonal spike). Detection of paraproteins in the urine or blood is most often associated with monoclonal gammopathy of undetermined significance (MGUS), a precursor of multiple myeloma and in multiple myeloma. An excess in the blood is known as paraproteinemia. Unlike normal immunoglobulin antibodies, paraproteins cannot fight infection.

Currently MM patients are treated with chemotherapy for the neoplasm with nothing available for AL amyloidosis deposition in the organ, yet their clinical symptoms and prognosis are associated with their organ amyloid deposition. The antibodies described herein are expected to remove the amyloid from the organs as all amyloid in these co-morbid lymphoproliferative disorders arise from misfolded immunoglobulin light chains, the target of the antibodies.

In one aspect, the subject is currently or has been previously treated for multiple myeloma. In some aspects, a treatment for multiple myeloma is selected from the group consisting of chemotherapy, corticosteroid, immunomodulating agent, proteasome inhibitor, histone deacetylase (HDCA) inhibitor, immunotherapy, nuclear export inhibitor, stem cell transplant, radiation therapy, surgery, and any combination thereof.

The term “chemotherapy” or “chemotherapeutic agent” as used herein refers to any therapeutic agent used to treat cancer. Chemotherapeutic agent can include any substance or agent having a toxic effect on cells resulting in cell death or reduced proliferation, and especially cancer cell death regardless of the cellular pathway leading to it. Chemotherapy that can be used for the treatment of multiple myeloma can include, melphalan (a chemotherapeutic agent used to treat multiple myeloma, ovarian cancer, melanoma, and amyloidosis), vincristine (oncovin), cyclophosphamide (Cytoxan, a chemotherapeutic agent that suppress the immune system), etoposide (vp-16), doxorubicin (adriamycin), liposomal doxorubicin (doxil), or bendamustine (treanda).

Cyclophosphamide can induce the formation of DNA crosslinks both between and within DNA strands at guanine N-7 positions in cells that have low levels of ALDH. DNA crosslinked are irreversible and lead to cell apoptosis. Cyclophosphamide induces beneficial immunomodulatory effects in adaptive immunotherapy, notably by eliminating T regulatory cells (CD4+CD25+ T cells).

Melphalan is orally or intravenously administered, and chemically alters DNA nucleotide guanine through alkylation. Alkylation causes linkages between strands of DNA that in turn inhibits DNA synthesis and RNA synthesis, and cause cytotoxicity in both dividing and non-dividing tumor cells. Common side effect of melphalan includes bone marrow suppression, which is beneficial for the treatment of amyloidosis.

Corticosteroids are a class of steroid hormones that are produced in the adrenal cortex of vertebrates, as well as the synthetic analogues of these hormones. Two main classes of corticosteroids, glucocorticoids and mineralocorticoids, are involved in a wide range of physiologic processes, including stress response, immune response, and regulation of inflammation, carbohydrate metabolism, protein catabolism, blood electrolyte levels, and behavior. Some common naturally occurring steroid hormones are cortisol, corticosterone, and cortisone. Other examples of corticosteroids include prednisone, prednisolone, dexamethasone, budesonide, beclomethasone dipropionate, triamcinolone acetonide, fluticasone propionate, fluticasone furoate, flunisolide, methylprendisone and hydrocortisone.

Corticosteroids, such as dexamethasone and prednisone are an important part of the treatment of multiple myeloma. They can be used alone or combined with other drugs as a part of treatment. Corticosteroids are also used to help decrease the nausea and vomiting that chemotherapy might cause. Dexamethasone is a corticosteroid medication used in the treatment of many conditions, including rheumatic problems, a number of skin diseases, severe allergies, asthma, chronic obstructive lung disease, croup, brain swelling, eye pain following eye surgery, and along with antibiotics in tuberculosis.

The term “immune modulator” or “immunomodulating agent” as used herein refers to any therapeutic agent that modulates the immune system. Examples of immune modulators include eicosanoids, cytokines, prostaglandins, interleukins, chemokines, checkpoint regulators, TNF superfamily members, TNF receptor superfamily members and interferons. Specific examples of immune modulators include PGI2, PGE2, PGF2, CCL14, CCL19, CCL20, CCL21, CCL25, CCL27, CXCL12, CXCL13, CXCL-8, CCL2, CCL3, CCL4, CCL5, CCL11, CXCL10, IL1, IL2, IL3, IL4, IL5, IL6, IL7, IL8, IL9, IL10, IL11, IL12, IL13, IL15, IL17, IL17, INF-α, INF-β, INF-ε, INF-γ, G-CSF, TNF-α, CTLA, CD20, PD1, PD1L1, PD1L2, ICOS, CD200, CD52, LTa, LTap, LIGHT, CD27L, 41BBL, FasL, 0x40L, April, TL1A, CD30L, TRAIL, RANKL, BAFF, TWEAK, CD40L, EDA1, EDA2, APP, NGF, TNFR1, TNFR2, LTOR, HVEM, CD27, 4-1BB, Fas, 0x40, AITR, DR3, CD30, TRAIL-R1, TRAIL-R2, TRAIL-R3, TRAIL-R4, RANK, BAFFR, TACI, BCMA, Fn14, CD40, EDAR XEDAR, DR6, DcR3, NGFR-p75, and Taj. Other examples of immune modulators include tocilizumab (Actemra®), CDP870 (Cimzia®), enteracept (Enbrel®), adalimumab (Humira®), Kineret®, abatacept (Orencia®), infliximab (Remicade®), rituzimab (Rituxan®), golimumab (Simponi®), Avonex®, Rebif®, Recigen®, Plegridy®, Betaseron®, Copaxone®, Novatrone®, natalizumab (Tysabri®), fingolimod (Gilenya®), teriflunomide (Aubagio®), BG12, Tecfidera®, and alemtuzumab (Campath®, Lemtrada®).

Immunomodulating agents that can be used to treat multiple myeloma include thalidomide, lenalidomide and pomalidomide.

Thalidomide (Thalomid®) was first used decades ago as a sedative and as a treatment for morning sickness in pregnant women. When it was found to cause birth defects, it was taken off the market but became available again as a treatment for multiple myeloma. Side effects of thalidomide can include drowsiness, fatigue, severe constipation, and painful nerve damage (neuropathy). The neuropathy can be severe and might not go away after the drug is stopped. There is also an increased risk of serious blood clots that start in the leg and can travel to the lungs.

Lenalidomide (Revlimid®) is similar to thalidomide. It works well in treating multiple myeloma. The most common side effects of lenalidomide are thrombocytopenia (low platelets) and low white blood cell counts. It can also cause painful nerve damage. The risk of blood clots is not as high as that seen with thalidomide, but it is still increased. In patients, where the myeloma is in remission after either a stem cell transplant or initial treatment, lenalidomide may be given for maintenance therapy to prolong the remission.

Pomalidomide (Pomalyst®) is also related to thalidomide and is used to treat multiple myeloma. Some common side effects include low red blood cell counts (anemia) and low white blood cell counts. The risk of nerve damage is not as severe as it is with the other immunomodulating drugs, but it's also linked to an increased risk of blood clots.

Proteasome inhibitors work by stopping enzyme complexes (proteasomes) in cells from breaking down proteins important for controlling cell division. They appear to affect tumor cells more than normal cells, but they are not without side effects. Proteasome inhibitor than can be used to treat multiple myeloma include bortezomib, carfilzomib and ixazomib.

Bortezomib (Velcade®) was the first of this type of drug to be approved and is often used to treat multiple myeloma. It may be especially helpful in treating myeloma patients with kidney problems. In patients where the myeloma was put into remission after either a stem cell transplant or initial treatment, bortezomib may also be given for maintenance therapy to prolong the remission.

Carfilzomib (Kyprolis®) is a newer proteasome inhibitor that can be used to treat multiple myeloma in patients who have already been treated with other drugs that didn't work. To prevent problems like allergic reactions during the infusion, the steroid drug dexamethasone is often given before each dose in the first cycle.

Ixazomib (Ninlaro®) is a proteasome inhibitor that is a capsule taken by mouth, typically once a week for 3 weeks, followed by a week off. This drug is usually given after other drugs have been tried.

Histone deacetylase (HDAC) inhibitors are a group of drugs that can affect which genes are active or turned on inside cells. They do this by interacting with proteins in chromosomes called histones. HDAC inhibitor that can be used for the treatment of multiple myeloma include panobinostat. Panobinostat (Farydak®) is an HDAC inhibitor that can be used to treat patients who have already been treated with bortezomib and an immunomodulating agent. It is a capsule, typically taken 3 times a week for 2 weeks, followed by a week off. This cycle is then repeated.

The term “immunotherapy” refers to any type of therapy that includes modulating the immune system or the immune response. Modulating the immune system includes inducing, stimulating or enhancing the immune system as well as reducing, suppressing or inhibiting the immune system. Immunotherapy can be active or passive. Passive immunotherapy relies on the administration of monoclonal antibodies directed against the target to eliminate. For example, tumor-targeted monoclonal antibodies have demonstrated clinical efficacy to treat cancer. Active immunotherapy aims to induce a cellular immunity and establish immunological memory against the target agent. Active immunotherapy includes but is not limited to vaccination and immune modulators. Immunotherapy that can be used for the treatment of multiple myeloma includes monoclonal antibodies such as anti-CD38 antibodies and anti-SLAMF7 antibodies, and antibody-drug conjugates.

Daratumumab (Darzalex®) is a monoclonal antibody that attaches to the CD38 protein, which is found on myeloma cells. This is thought to both kill the cancer cells directly and to help the immune system attack them. This drug is used mainly in combination with other types of drugs, although it can also be used by itself in patients who have already had several other treatments for their myeloma. A newer form of the drug, known as daratumumab and hyaluronidase (Darzalex® Faspro®), can be given as a subcutaneous (under the skin) injection, typically in the belly area over a few minutes. Isatuximab (Sarclisa®) is another monoclonal antibody that attaches to the CD38 protein on myeloma cells. This is thought to both kill the cancer cells directly and to help the immune system attack them. This drug is used along with other types of myeloma drugs, typically after at least 2 other treatments have been tried.

Elotuzumab (Empliciti®) is a monoclonal antibody that attaches to the SLAMF7 protein, which is found on myeloma cells. This is thought to help the immune system attack the cancer cells. This drug is used mainly in patients who have already had other treatments for their myeloma.

The term “antibody-drug conjugate” as used herein refers to a monoclonal antibody linked to a chemotherapy drug. Antibody-drug conjugate for the treatment of multiple myeloma include an antibody targeting BCMA protein on myeloma cells, and a chemotherapeutic agent. Belantamab mafodotin-blmf (Blenrep®) is an antibody-drug conjugate that can be used by itself to treat myeloma mainly in people who have already had at least 4 other treatments for their myeloma (including proteasome inhibitors, immunomodulatory drugs, and a monoclonal antibody to CD38).

“Nuclear export inhibitor”, or “selective inhibitors of nuclear export” (SINEs) are drugs that block exportin 1 (XPO1 or CRM1), a protein involved in transport from the cell nucleus to the cytoplasm. This inhibition causes cell cycle arrest and cell death by apoptosis, and SINE compounds are of interest as anticancer drugs. Selinexor (Xpovio®) has been approved for treatment of multiple myeloma as a drug of last resort. It is usually used with dexamethasone.

“Stem cell transplant” or “bone marrow transplant”, as used herein, refers to the depletion of a patient of all the cells in his bone marrow (including cancer cells such as myeloma cells) using high-dose chemotherapy, and the transplant of new, healthy blood-forming stem cells. Stem cell transplant is commonly used to treat multiple myeloma. The transplant can either be autologous, using the patient's own stem cells removed from his or her bone marrow or peripheral blood before the transplant; or allogenic, using blood-forming stem cells from a donor matched to the patient's cell type (such as a close relative to the patient, such as a brother or sister). Stem cell transplant is a standard treatment for patients with multiple myeloma. Although an autologous transplant can make the myeloma go away for a time (even years), it doesn't cure the cancer, and often the myeloma returns.

Radiation may be used to treat areas of bone damaged by myeloma that have not responded to chemotherapy and/or other drugs and are causing pain or may be near breaking. It's also the most common treatment for solitary plasmacytomas.

Surgery is sometimes used to remove single plasmacytomas, but it's rarely used to treat multiple myeloma. When spinal cord compression causes paralysis, severe muscle weakness, or numbness, emergency surgery may be needed. Surgery to attach metal rods or plates can help support weakened bones and may be needed to prevent or treat fractures.

All the additional treatment described herein, that can be used for the treatment of multiple myeloma can be used alone or in various combination. Among those combinations, the following combinations are often used for the treatment of multiple myeloma:
Lenalidomide (or pomalidomide or thalidomide) and dexamethasone;
Carfilzomib (or ixazomib or bortezomib), lenalidomide, and dexamethasone;
Bortezomib (or carfilzomib), cyclophosphamide, and dexamethasone;
Elotuzumab (or daratumumab), lenalidomide, and dexamethasone;
Bortezomib, liposomal doxorubicin, and dexamethasone;
Panobinostat, bortezomib, and dexamethasone;
Elotuzumab, bortezomib, and dexamethasone;
Melphalan and prednisone (MP), with or without thalidomide or bortezomib;
Vincristine, doxorubicin (Adriamycin), and dexamethasone (called VAD);
Dexamethasone, cyclophosphamide, etoposide, and cisplatin (called DCEP);
Dexamethasone, thalidomide, cisplatin, doxorubicin, cyclophosphamide, and etoposide (called DT-PACE), with or without bortezomib;
Selinexor, bortezomib, dexamethasone,
Idecabtagene vicleucel, a B-cell maturation antigen-directed chimeric antigen receptor (CAR) T-cell therapy.

The choice and dose of drug therapy depend on many factors, including the stage of the cancer, the age and kidney function of the patient as well as how frail the patient may be. If a stem cell transplant is planned, most doctors avoid using certain drugs, like melphalan, that can damage the bone marrow.

In another embodiment, the invention provides a method of treating amyloidosis in a subject having a plasma cell disease including administering to the subject a pharmaceutical composition including an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains, thereby treating the plasma cell disease in the subject.

Plasma cell disorders are a diverse group of disorders of unknown etiology characterized by a disproportionate proliferation of a single clone of B cells and by the presence of a structurally and electrophoretically homogeneous (monoclonal) immunoglobulin or polypeptide subunit in the serum, urine, or both. After developing in the bone marrow, undifferentiated B cells normally enter peripheral lymphoid tissues, such as lymph nodes, spleen, and gut (e.g., Peyer patches) where they begin to differentiate into mature cells, each of which can respond to a limited number of antigens. After encountering the appropriate antigen, some B cells undergo proliferation into plasma cells. Each plasma cell line is committed to synthesizing one specific immunoglobulin antibody that consists of 2 identical heavy chains (gamma [γ], mu [μ], alpha [α], delta [δ], or epsilon [ε]) and 2 identical light chains (kappa [κ] or lambda [λ]). A slight excess of light chains is normally produced, and urinary excretion of small amounts of free polyclonal light chains (≤40 mg/24 hours) is normal. Plasma cell disorders are of unknown etiology and are characterized by the disproportionate proliferation of one clone. The result is a corresponding increase in the serum level of its product, the monoclonal immunoglobulin protein (M protein), which can consist of both heavy and light chains or of only one type of chain.

Plasma cell disorders can be classified in 2 categories: (1) monoclonal gammopathy ofundetermined significance, which are usually asymptomatic and associated with monoclonal B or plasma cells, with chronic inflammatory and infection conditions (including chronic cholecystitis, osteomyelitis, pyelonephritis, rheumatoid arthritis, and tuberculosis), or associated with other disorders (including familial hypercholesterolemia, Gaucher disease, Kaposi sarcoma, lichen myxedematous, liver disorders, myasthenia gravis, pernicious anemia and hyperthyroidism); and (2) malignant plasma cell disorders, which can either be asymptomatic such as (a) smoldering multiple myeloma, (b) symptomatic and active multiple myeloma associated with immunoglobulin and/or light chains production, (c) primary systemic amyloidosis associated with monoclonal light chains (nonhereditary), or associated with heavy chains (IgG, IgA, IgM or IgD heavy chain disease) (d) B-cell lymphoma associated with production of a monoclonal protein.

The most common plasma cell diseases include monoclonal gammopathy of undetermined significance (MGUS, which along smoldering multiple myeloma is a plasma cell disease in which patients are not yet sick because they have very limited organ damage, if any), multiple myeloma, and systemic light-chain (AL) amyloidosis. Plasma cell proliferation and M protein production are associated with various symptoms of the diseases including: (1) damage to organs, and particularly the kidneys due to hypercalcemia or toxic light chains secreted by the malignant plasma cell, and due to the fact that some M proteins show antibody activity against self-antigens; (2) impaired immunity, due to the decreased production of other immunoglobulins; (3) bleeding tendency, due to the ability of M protein to coat platelets, inactivate clotting factors, and increase blood viscosity; (4) amyloidosis, due to the ability of M protein and or light chains to form fibrillar deposits within organs (most commonly the heart, kidney and liver); and (5) osteoporosis, hypercalcemia, anemia, or pancytopenia, due to the over-activation of osteoclasts by monoclonal plasma cells in bone matrix and/or marrow.

In one aspect, the plasma cell disease is selected from the group consisting of low-grade B-cell lymphoma, monoclonal gammopathy of undetermined significance (MGUS), and multiple myeloma.

In some aspects, the subject is currently or has been previously treated for the plasma cell disease. In other aspects, a treatment for plasma cell disease includes chemotherapy.

In an additional embodiment, the invention provides a method of identifying a subject having multiple myeloma as a candidate for an anti-amyloidosis treatment comprising identifying light chain (AL) amyloidosis fibrils and/or amyloid protein precursor deposition in the subject, wherein the identification of AL amyloidosis fibrils and/or amyloid protein precursor deposition in the subject is indicative of the likelihood of the subject to respond to the anti-amyloidosis treatment, and wherein the anti-amyloidosis treatment includes an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains, thereby identifying the subject as a candidate for the anti-amyloidosis treatment.

As used herein, identifying AL amyloidosis fibrils and/or amyloid protein precursor deposition in the subject can include subjecting the subject to any methods of diagnostic of amyloidosis known in the art. Amyloidosis can be detected in a subject using laboratory tests, biopsies, and/or imaging tests.

Laboratory tests can include blood and urine analysis for the detection of abnormal protein that can indicate amyloidosis. Depending on the signs and symptoms, thyroid and liver function tests may also be indicated. Blood and urine tests can also aid in discovering which organs are involved and how much they are compromised. For example, a 24-hour urine collection to look at the level of protein in your urine sample can indicate excess protein in the urine which may be an indication of kidney involvement. Blood test can also be used to test for the presence of abnormal antibody (immunoglobulin) proteins in the blood (to evaluate the level of kappa and lambda light chains).

A tissue biopsy involves the removal of a small sample of tissue to find evidence of amyloid deposits. Any kind of tissue or organ biopsy can be stained with a “Congo-red stain” and analyzed to detect amyloidosis deposits. Less invasive biopsies include fat pad biopsy (from under the skin in the abdomen); labial salivary gland biopsy (the inner lip); and, skin or bone marrow. Bone marrow tests can include bone marrow aspirate (involving the removal of some liquid bone marrow) and bone marrow biopsy (involving the removal of a 1-2 cm core of bone marrow tissue in one piece). These samples can help to determine the percentage of amyloid producing plasma cells, and when tested in the lab they can assist in identifying whether the abnormal plasma cells are producing kappa or lambda light chains. More invasive biopsy can include organ biopsy, usually performed if amyloidosis is suspected but biopsies of the bone marrow, fat pad, lip or skin sites turn up negative. A surgical biopsy of the organ that is indicating symptoms can then be performed in the liver, kidney, nerve, heart or gut (stomach or intestines).

Imaging test can include echocardiogram and other imaging, that can be used to help establish the extent of the disease. Using an echocardiogram amyloid deposits can be detected in the heart, while viewing the size and shape of it and the location and extent of any impact of amyloid. Other imaging can include MRI (magnetic resonance imaging), and, CMR (for cardiac magnetic resonance), pyrophosphate scanning (a nuclear medicine test also used to evaluate whether an unusual type of cardiomyopathy is present). Nuclear imaging, using radioactive tracers injected to the subject can also be used to reveal early heart damage caused by certain types of amyloidosis. It can also help distinguish between different types of amyloidosis, which can guide treatment decisions. The antibody described herein can also be used for imaging purposes, when coupled to a radioactive tracer such as ¹²⁴I to generate a tagged antibody. Such imaging technique can provide both a localization and extend of deposited amyloid fibrils in the subject. Thus, a labeled antibody of the disclosure may be used to detect the presence of amyloid deposition disease in a patient suspected of having the disease as well as to determine the effectiveness of treatment.

In one aspect, the subject is currently or has been previously treated for multiple myeloma.

In another aspect, the method further includes administering to the subject a treatment for multiple myeloma. In some aspects, a treatment for multiple myeloma is selected from the group consisting of chemotherapy, corticosteroid, immunomodulating agent, proteasome inhibitor, histone deacetylase (HDCA) inhibitor, immunotherapy, nuclear export inhibitor, stem cell transplant, radiation therapy, surgery, and any combination thereof.

The antibody of the present disclosure can be administered to any subject having amyloidosis, whether or not their amyloidosis is hematologically controlled or not. As used herein, the description of “not hematologically controlled” with respect to AL amyloidosis means that the disease is not in either complete remission or very good partial remission. For example, the disease is not hematologically controlled when the patient has detectable levels of toxic amyloid precursor proteins in circulation (i.e., serum or urine) or when the difference between involved and uninvolved free light chains is >40 mg/L in the patient's blood or serum. For example, the subject can have an hematologically controlled amyloidosis, and therefore doesn't have a hematologic disease. The subject can also have an hematologically uncontrolled amyloidosis, and therefore have a hematologic disease. Both serum and urine protein electrophoresis (SPEP and UPEP, respectively) along with serum and urine immunofixation (SIF and UIF, respectively) can be obtained for both diagnosis and monitoring of an amyloidosis disease. For example, as a standard of care, SPEP, UPEP, SIF, and/or UIF can be evaluated every 3 months.

In one aspect, the subject has a hematologic disease. A hematologic disease can, for example, be characterized by the measure of the involved/uninvolved free light chain difference (dFLC), by a measure a SPEP or a UPEP m-spike

In one aspect, the hematologic disease is characterized by an involved/uninvolved free light chain difference (dFLC) >5 mg/dL or by a FLC >5 mg with abnormal ratio.

In another aspect, the hematologic disease is characterized by a serum protein electrophoresis (SPEP) or a urine protein electrophoresis (UPEP) m-spike >0.5 g/dL.

In one aspect, the disclosure provides a method of treating an amyloidosis disease or disorder involving a kidney, a gastrointestinal tract, liver or a heart in a subject including administering to the subject the composition described herein.

When an amyloid disease affects the heart, it can cause numerous types of complications. As amyloid deposits or plaques reduce the heart's ability to fill with blood between heartbeats, cardiac involvement is associated with poor prognosis. Less blood is pumped with each beat, and this may lead to shortness of breath. Amyloid deposits or plaques in or around the heart may also cause irregular heartbeats and congestive heart failure, among other organ dysfunctions.

As used herein, “treating an amyloidosis involving the heart” refers to the reduction, amelioration, improvement, reversal and the like of any symptoms associated with the deposit of amyloid fibrils in the heart or of any cardiac function or parameter impacted by the deposit of amyloid fibrils in the heart. As used herein, “cardiac involvement” means that a patient suffering from an amyloid disease have amyloid deposits in the heart. Amyloid deposits in the heart result in release of NT-proBNP and increased NT-proBNP levels in the blood of the patient. Herein, a patient has cardiac involvement if NT-proBNP is greater than 650 pg/mL. A patient's cardiac involvement can also be evaluated by elevated cardiac troponin (cTn) levels. For example, a patient has a cardiac involvement if cTnT level is less than 0.035 g/L

Myocardial function and improvement therein can be measured by using echocardiography to measure global longitudinal strain (GLS) as described in Smiseth et al.—Eur Heart J, 37:1196. Echocardiography uses ultrasound to measure the average deformation within segments of the myocardium, and GLS is the average of these segments as a measure of global left ventricular function. Amyloid deposits can result in thickened left and right ventricular walls and in a non-dilated ventricle that is stiff and poorly compliant, resulting in “strain” on the heart and vasculature. In echocardiography parlance, the term “strain” is used to describe deformation in the myocardium, which may include but is not limited to local shortening, thickening, and/or lengthening of the myocardium. Strain can be used as a measure of ventricular function. A person having ordinary skill in the art will know how to use echocardiography to determine GLS and will understand that it may be calculated in various ways. For example, the Lagrangian formula (ε_{L}=(L-L₀)/L₀=ΔL/L₀, where L₀ is baseline length and L is the resulting length), defines strain in relation to the original length as a dimensionless measure, in which shortening will be negative, and lengthening will be positive. It is usually expressed in percent. An alternative definition, Eulerian strain, defines the strain in relation to the instantaneous length: ε_{E}=ΔL/L. For a change over time, the Lagrangian strain will be: ε_{L}=ΣΔL/L₀, and Eulerian Strain ε_{E}=Σ(ΔL/L). The term was first used by Mirsky and Parmley in describing regional differences in deformation between normal and ischemic myocardium.

Hence, in some aspects, the patients of the presently disclosed method exhibits an improvement in global longitudinal strain (GLS) compared with pretreatment GLS level upon administration of the antibody. In some aspects, a patient with cardiac involvement may have a baseline NT-proBNP of at least 650 pg/ml, while in other aspects, a patient with cardiac involvement may have a baseline NT-proBNP of at least 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, or 2300 or more pg/ml.

In some aspects, the improvement in GLS may occur within about 1, about 2, about 3, about 4, about 5, about 6, about 7, about 8, about 9, about 10, about 11, about 12, about 13, about 14, or about 15 weeks of the initiation of treatment. In some embodiments, the improvement in GLS may be represented by a 1, 2, 3, 4, 5, 6, 7, 8, 9, 10, 11, 12, 13, 14, 15, 16, 17, 18, 19, 20, 21, 22, 23, 24, 25, or more percent reduction in GLS compared to baseline as calculated by the Lagrangian formula. A reduction in GLS level compared to baseline of about 2% percent or more is considered clinically relevant. In some aspects, the disclosed treatments with the antibody may decrease the levels of the patient's N-terminal pro b-type natriuretic peptide (NT-proBNP) by at least about 30% compared to baseline levels taken prior to administration of the antibody. In some aspects, the decrease in NT-proBNP may be at least about 40%, at least about 50%, at least about 60% or more compared to baseline levels taken prior to administration of the antibody. In other aspects, treatment with the disclosed antibody may result in the patient's NT-proBNP level decreasing to less than about 9100 ng/L following administration of the antibody. In other embodiments, the patient's NT-proBNP level may decrease to less than about 8000, 7000, 6000, 5000, or 4000 ng/L following administration of the antibody. In some aspects, the patient may initially be classified as New York Heart Association (NYHA) Functional Classification class II or III prior to administration of the antibody, but after treatment with the disclosed antibody the patient may be classified as class I on the NYHA classification scale. However, when renal function declines, such that eGFR is less than 30 for example, NT-proBNP is not used but BNP is measured instead.

Immunoglobulins are composed of four protein chains: two light chains, either kappa (κ) or lambda (λ) light chains, and two heavy chains, of which there are several types. In AL amyloidosis, either kappa light chains or lambda light chain may be misfolded and form amyloid fibrils or plaques. Hence, in some patients, both kappa and lambda fragments may be misfolded. Subgroup analysis showed that patients with both lambda cardiac involvement and kappa cardiac involvement showed improvement by having reduced GLS compared to pretreatment levels. Hence, in some aspects, the patient is further characterized as having light chain lambda amyloid cardiac involvement. In other aspects, the patient is further characterized as having light chain kappa amyloid cardiac involvement.

The disclosed methods of treatment may be particularly beneficial for patients with disease that is not hematologically controlled (i.e., in neither complete remission nor very good partial remission) because the presently disclosed antibodies are believed to be able to bind to and neutralize toxic amyloid precursor proteins in circulation, even before the proteins aggregate to form an amyloid deposit. Complete remission is defined as negative serum and urine immunofixation and normal ratio in a free-light-chain (FLC) assay, while very good partial remission is defined as having a difference between involved and uninvolved free light chains of <40 mg/L.

Echocardiography is non-invasive and can be used to monitor improvement in myocardial function in a patient diagnosed with light chain amyloidosis (ALA) having a cardiac involvement including observing an improvement in myocardial function in a patient diagnosed with ALA having a cardiac involvement within about three weeks after administration to said patient of a therapeutically effective amount of an antibody. In some aspects, the improvement in myocardial function persists for a period extending at least three months after administration of the antibody.

When an amyloid disease affects the kidneys, it will often harm the kidneys' filtration ability, allowing protein to leak from the blood into the urine (i.e., proteinuria). Moreover, the kidneys' ability to remove waste products from the body is lowered, which may eventually lead to kidney failure.

In some aspects, when the disease involves amyloid deposits or plaques in the patient's kidneys, treatment with the disclosed antibody may decrease proteinuria by at least about 30% compared to baseline levels determined prior to administration of the antibody. In some aspects, the decrease in protein in the patient's urine may be at least about 40%, at least about 50%, at least about 60% or more compared to baseline levels determined prior to administration of the antibody. In some embodiments, the patient's urine protein output may decrease to less than about 7000, less than about 6000, less than about 5000, less than about 4000, or less than about 3000 mg/24 hours following administration of the antibody.

As used herein, “treating an amyloidosis involving the kidney” refers to the reduction, amelioration, improvement, reversal and the like of any symptoms associated with the deposit of amyloid fibrils in the kidney or of any renal function or parameter impacted by the deposit of amyloid fibrils in the kidney.

When an amyloid disease affects the gastrointestinal (GI) tract, it will often harm the GI tract ability to absorb nutrients. Loss of proper functioning of the GI tract due to amyloid deposits may can lead to weight loss, diarrhea, abdominal pain, malabsorption, esophageal reflux, and varying degrees of upper and lower GI bleeding, including fatal hemorrhage. Hepatic symptoms include jaundice, steatorrhea, anorexia, and those related to portal hypertension such as ascites and splenomegaly.

As used herein, “treating an amyloidosis involving the gastrointestinal tract” refers to the reduction, amelioration, improvement, reversal and the like of any symptoms associated with the deposit of amyloid fibrils in the GI tract.

For example, when an amyloidosis disease affects the liver, it can induce abdominal distention, hepatomegaly, ascites, and/or oliguria. Other symptoms may include but are not limited to changes in alkaline phosphatase (ALP) levels and/or serum y-glutamyltransferase (GGT) levels, hyperlipidemia, coagulation abnormalities, thrombocytopenia, prothrombin time (PT), erythrocyte sedimentation rate, alanine aminotransferase (ALT) and/or aspartate aminotransferase (AST), serum albumin and complement fragment levels. Conventional imaging such echography, computed tomography scanner, X-ray, endoscopy and the like, coupled with blood analysis and physical examination can be used to monitor any change, amelioration or improvement of the symptoms associated with amyloid deposits in the liver.

In various aspects, an additional therapy is further administered to the subject.

In some aspects, the additional therapy includes cyclophosphamide, bortezomib, dexamethasone, daratumumab, melphalan, lenalidomide, isatuximab, venetoclax, a stem cell transplant or a combination thereof.

In an additional embodiment, the disclosure provides a method of treating one or more symptoms of an amyloidosis disease or disorder involving the skin in a subject including administering to the subject the pharmaceutical composition of the present disclosure.

In one aspect, the one or more symptoms of the amyloidosis disease or disorder involving the skin include hair loss, facial hair loss and body hair loss.

In one aspect, an additional therapy is further administered to the subject.

In some aspects, the additional therapy includes cyclophosphamide, bortezomib, dexamethasone, daratumumab, melphalan, lenalidomide, isatuximab, venetoclax, a stem cell transplant or a combination thereof.

In one embodiment, the disclosure provides a method of inhibiting and/or reducing aggregation of light chains and amyloid fibrils in a subject including administering to the subject about 1,000 mg/m² of an antibody having a heavy chain variable domain (VH) having an amino acid sequence of SEQ ID NO:1 and a light chain variable domain (VL) having an amino acid sequence of SEQ ID NO.2.

As used herein “inhibiting and/or reducing aggregation of light chains and amyloid fibrils” may interchangeably refer to both facilitating the removal of existing amyloid deposit, and to preventing the formation of new ones through the de novo aggregations of light chains and amyloid fibrils.

In another embodiment, the disclosure provides a method of treating an amyloidosis disease or disorder in a subject including administering to the subject the pharmaceutical composition described herein.

In one aspect, administering the pharmaceutical composition renders the subject eligible for a stem cell transplant.

Stem cell transplantation or bone marrow transplantation is considered the most efficient treatment for patients suffering from an amyloidosis disease or disorder as the plasma cells in the bone marrow that produce amyloid protein are first destroyed by high doses of chemotherapy and then replaced by hematopoietic stem cells from a donor, that develop into healthy bone marrow. Survival can be significantly improved with high-dose chemotherapy and peripheral blood stem cell transplantation. However, to be eligible for a stem cell transplant, a subject must have some normal organ function; therefore, many patients cannot receive this treatment because the amyloid protein buildup has affected the function of other organs. By decreasing the amount, size and/or ability of amyloid fibrils to aggregate, the antibody of the present disclosure can reduce organ disfunction, which is one of the main obstacle to stem cell transplant, and place the patient in a favorable disposition to be a recipient candidate for a stem cell transplant.

In other aspect, a stem cell transplant is further performed in the subject.

Therapeutically effective doses and dosing regimens of the foregoing methods may vary, as would be readily understood by those of skill in the art. Dosage regimens may be adjusted to provide the optimum desired response (e.g., a therapeutic response clearance of amyloid plaques or reduction in the amount of deposited amyloid fibrils).

In one aspect, the antibody is administered weekly for at least 2, 3 or 4 weeks. Administration of antibody of the disclosure is considered the loading dose, which is the initial dose administered to a subject. The loading dose can for example be followed by a maintenance dose.

In one aspect, a maintenance dose of antibody is further administered to the subject thereafter.

The maintenance dose can be administered at a regimen that is similar to the regimen followed during the loading dose, or the maintenance dose can be administered at a distinct regimen as compared to the regimen followed during the loading dose. For example, the maintenance dose can be administered less often that the loading dose.

In some aspects, the maintenance dose is administered biweekly, triweekly, or monthly after the first 2, 3, 4 or more weeks of weekly administration.

Various other administration regimen may be suitable for the methods described herein. For example, in some aspects, a single dose of the antibody may be administered, while in other aspects, several divided doses may be administered over time, or the dose may be proportionally reduced or increased in subsequent dosing as indicated by the situation. For example, in some aspects, the disclosed antibodies may be administered once or twice weekly by subcutaneous, intravenous, or intramuscular injection. In some aspects, the disclosed antibodies may be administered once or twice monthly by subcutaneous, intravenous, or intramuscular injection. In some aspects, the disclosed antibodies may be administered once or twice annually by subcutaneous, intravenous, or intramuscular injection. In other aspects, the disclosed antibodies or antigen-binding fragments thereof may be administered once every week, once every other week, once every three weeks, once every four weeks, once every month, once every other month, once every three months, once every four months, once every five months, once every six months, once every seven months, once every eight months, once every nine months, once every ten months, once every eleven months, twice a year, or once a year, as the situation or condition of the patient may indicate.

As further detailed in the examples, the treatment with the antibody is not only sustained, but rapid as well. In some aspects, a patient may experience a therapeutic response (i.e., a decrease in the size of amyloid deposits or plaques, a decrease in the rate of plaque formation, or improved organ function) in one week or less, two weeks or less, three weeks or less, four weeks or less, five weeks or less, six weeks or less, seven weeks or less, eight weeks or less, nine weeks or less, ten weeks or less, eleven weeks or less, twelve weeks or less, or any time frame in between. For example, depending on the dose and dosing regimen, a patient may experience a therapeutic response in about a week or about 4.5 weeks.

The therapeutically effective dose of antibody administered to the patient (whether administered in a single does or multiple doses) should be sufficient to reduce the amount of deposited amyloid fibrils in the patient. Such therapeutically effective amount may be determined by evaluating the symptomatic changes in the patient or by evaluating the change in the amount of deposited amyloid fibrils (e.g., by radioimmune detection of deposited amyloid deposits using ¹²⁴I tagged antibody). Thus, a labeled antibody of the disclosure may be used to detect the presence of amyloid deposition disease in a patient suspected of having the disease as well as to determine the effectiveness of treatment.

Exemplary doses can vary according to the size and health of the individual being treated, as well as the condition being treated. In some aspects, a therapeutically effective amount of a disclosed antibody may be from about 500 mg/m² to 1000 mg/m²; however, in some situations the dose may be higher. For instance, in some embodiments, the therapeutically effective amount may be about 1000, about 975, about 950, about 925, about 900, about 875, about 850, about 825, about 800, about 775, about 750, about 725, about 700, about 675, about 650, about 625, about 600, about 575, about 550, about 525, or about 500 mg/m².

In one aspect, an antibody administration dose is from about 500 mg/m² to 1,000 mg/m². In many aspects, the antibody administration dose is selected from about 500 mg/m², about 750 mg/m² and about 1,000 mg/m².

Similarly, in some aspects, the effective amount of an antibody is about 2,200 mg; however, in some situations the dose may be higher or lower. In some embodiments, a therapeutically effective amount may be between 50 and 5000 mg, between 60 about 4500 mg, between 70 and 4000 mg, between 80 and 3500 mg, between 90 and 3000 mg, between 100 and 2500 mg, between 150 and 2000 mg, between 200 and 1500 mg, between 250 and 1000 mg, or any dose in between. For instance, in some embodiments, the therapeutically effective amount may be about 50 about 60, about 70, about 80, about 90, about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000 or more mg.

In one aspect, administering a weekly dose of about 1,000 mg/m² of an antibody includes administering about 2,750 mg of the antibody.

In other aspects, administering a weekly dose of about 500 mg/m² of an antibody includes administering about 1,375 mg of the antibody, and administering a weekly dose of about 750 mg/m² of an antibody includes administering about 2,065 mg of the antibody.

Similarly, in some aspects, the effective amount of an antibody is about 25 mg/kg; however, in some embodiments, the concentration may be higher or lower. In some embodiments, the effective amount may be about 1-50 mg/kg, about 5-40 mg/kg, about 10-30 mg/kg, or about 15-25 mg/kg or any value in between. For instance, in some embodiments, the effective amount may be 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, 41, 42, 43, 44, 45, 46, 47, 48, 49, or 50 or more mg/kg. As shown in Example 11, the Phase 3 study results provided for a range (min/max) and the median doses, e.g., the median dose was 25.6 mg/kg, ranging from 19.8 to 31.1 mg/kg, with the vast majority of patients receiving between 22 and 28 mg/kg. (see Table 17).

In one aspect, administering about 1,000 mg/m² of an antibody includes administering about 25 mg/kg of the antibody.

In other aspects, an administration dose of about 500 mg/m² of an antibody includes about 12.5 mg/kg of the antibody, and an administration dose of about 750 mg/m² includes about 18.75 mg/kg of the antibody.

The disclosed methods of treatment may also be combined with other known methods of treatment as the situation may require. The current standard of care for AL amyloidosis, for example, generally involves autologous blood stem cell transplantation (ASCT) or an autologous bone marrow transplant. Thus, in some aspects, the disclosed antibodies may be administered prior to, after, or concurrently with other known treatments. In some aspects, the disclosed antibodies may be administered only after other treatment options have failed or the disease has continued to progress. In other words, in some embodiments, the disclosed antibodies are used to treat refractory amyloid disease, such as refractory AL amyloidosis.

As discussed above, the antibody can be administered in combination with an additional therapy. In one aspect, the antibody is administered prior to, simultaneously with, or after the additional therapy.

In various aspects, the antibody is administered before the additional therapy.

The methods described herein rely on the administration of the antibody of the present disclosure. In various aspects, administering the antibody to a subject includes administering to the subject a pharmaceutical composition including the antibody, sodium acetate, sodium chloride, mannitol, and polysorbate 80.

Beside its efficacy to treat a disease of interest, a therapeutic medication can be associated with other events not related to the treatment of such disease, which can include side effects, toxicities, or adverse events. For example, therapeutic medication can be associated with dose-limiting toxicity, when an increase of the dose is associated with an increase in the observed toxicity, which can limit or prohibit the use of therapeutically effective doses.

Some therapy may be associated with treatment-emergent adverse events (TEAEs), that were not present prior to the initiation of the treatment or that worsened in either intensity or frequency following exposure to the treatment. Common TEAEs include but are not limited to nausea, diarrhea, urinary trac infection, pain, dizziness, headache, fatigue and insomnia. As used herein, the term “serious adverse event” means an untoward medical event that results in death, is life-threatening, requires inpatient hospitalization or prolongation of existing hospitalization, or results in persistent or significant disfigurement or disability.

For example, the antibody of the present disclosure may be associated with event unrelated to the treatment of amyloidosis.

In one aspect, the 500 mg/m², 750 mg/m² and 1,000 mg/m² administration doses of antibody do not induce drug-related adverse events.

In another aspect, the 500 mg/m², 750 mg/m² and 1,000 mg/m² doses of antibody do not induce dose limiting toxicities.

The efficacy of the antibody to treat amyloidosis can also be measured based on pharmacokinetics parameters of the antibody.

For example, the efficacy of an antibody dose can be measured as its ability to bind its target (e.g., an amyloid deposit). The amyloid deposits can include aggregates of λ-light chain fibrils and/or κ-light chain fibrils.

In one aspect, the 500 mg/m², 750 mg/m² and 1,000 mg/m² administration doses induce binding of the antibody to the amyloid deposits.

The efficacy of an antibody dose can be measured as the site occupancy of the target receptor by the antibody. The site occupancy of a target receptor can indicate for a given dose of the antibody, which proportion of the amyloid deposits are bound to the antibody, and therefore actively target for degradation.

The administration doses of the antibody described herein can for example be sufficient to induce at least 50% occupancy of the target. The antibody can induce at least 50%, 60%, 70%, 80%, 90%, 95%, 96%, 97%, 98%, 99% or more occupancy of the amyloid deposits in a subject.

In one aspect, the 500 mg/m², 750 mg/m² and 1,000 mg/m² administration doses of antibody achieve a site occupancy of a target receptor of at least 90%.

The efficacy of an antibody dose can be measured as its concentration as measured in the subject, as compared to the administration dose.

In one aspect, the concentration of an antibody in the subject increases with the administration dose.

The efficacy of an antibody dose can be measured as the ability to efficiently remove amyloid deposits from a subject.

In one aspect, the amyloid deposits include aggregates of λ-light chain fibrils and/or κ-light chain fibrils. In other aspects, administering the antibody induces removal of amyloid deposits present in an organ or tissue. In various aspects, the organ or tissue is selected from the group consisting of heart, kidney, liver, lung, gastrointestinal tract, nervous system, muscular skeletal system, soft tissue and skin.

The following examples are given to illustrate the present disclosure. It should be understood, however, that the disclosure is not to be limited to the specific conditions or details described in these examples. All printed publications referenced herein are specifically incorporated by reference.

Presented below are examples discussing the efficacy of high doses of the antibody of the present disclosure, alone or in combination with a plasma cell directed therapy, contemplated for the discussed applications. The following examples are provided to further illustrate the embodiments of the present disclosure, but are not intended to limit the scope of the disclosure. While they are typical of those that might be used, other procedures, methodologies, or techniques known to those skilled in the art may alternatively be used.

### EXAMPLES

### Example 1

### Antibody Production and Characterization

The antibody of the disclosure was produced by transfecting host cell with a plasmid encoding a codon-optimized DNA sequence to improve translation efficiency a improve transcription efficiency, without altering the amino acid sequence of the antibody.

Cells were cultured in conditions to reach high antibody titer and cell density. The manufacturing process included production in a bioreactor until the optimal balance of cell debris/harvestability and antibody titer was reached.

The antibodies obtained were then characterized by studying charge heterogeneity under various stress conditions.

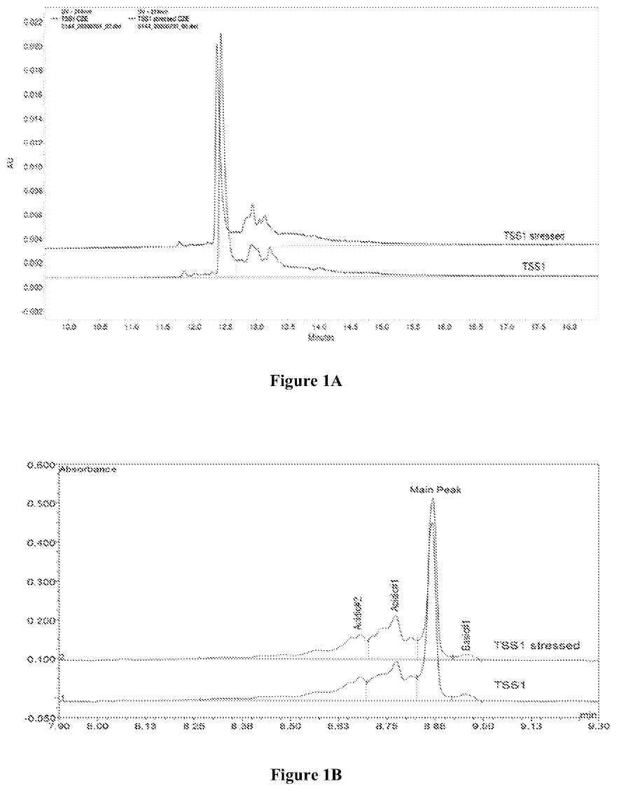

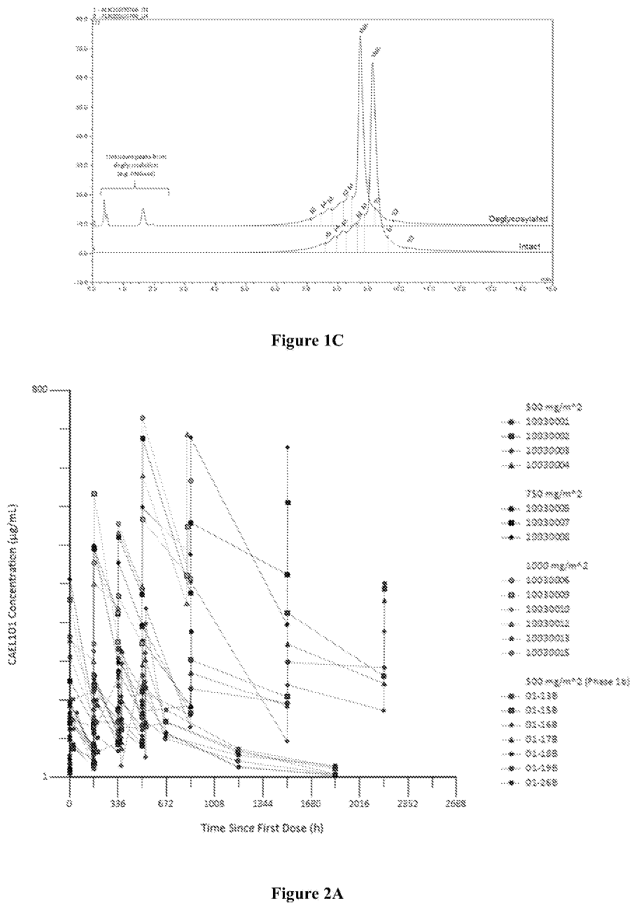

As illustrated in FIG. 1, the analysis of native, reduced, and reduced+deglycosylated fractions showed that all fractions were complex mixtures. All native fractions contained mixtures of the expected glycosylation variants.

AV4 & AV5 fractions contained the sialylated species. The main peak and BV1 fractions were enriched for the smaller neutral species (G0 & G0F). It was also shown that more acid fractions were enriched for galactosylated neutral species (G1F & G2F), and that the native AV5 fraction was enriched for halfmers (HC/LC), antibody missing N-terminal half of one HC, and other unidentified fragments. The native BV1 fraction was enriched for HC retaining C-terminal lysine, as expected; and the reduced AV3-5 LC fractions were enriched for glycated lysines.

As illustrated in FIG. 1, the antibody charge heterogeneity was assessed by capillary zone electrophoresis (CZE) separation (FIG. 1A), by capillary isoelectric focusing (cIEF) separation (FIG. 1B) and by cation exchange chromatography (CEX, FIG. 1C). The results shown that the apparent heterogeneity was not an artifact of a given method.

### Example 2

### Phase 1A/B Study

Relapsed or refractory AL Amyloidosis patients who received prior anti-plasma cell treatment were enrolled. Patients received the disclosed antibody as a single intravenous infusion (phase 1a) or a series of weekly infusions for 4 weeks (phase 1b). Briefly, the disclosed antibody is an IgG1 monoclonal antibody that targets the misfolded light chains of AL amyloid fibrils, a hallmark of AL amyloidosis. The disclosed antibody specifically binds to a conformational epitope present on both human kappa (κ) or lambda (λ) light chain amyloid fibrils. A dose-escalation “up and down” design was used for both phase 1a and 1 b where successive doses of 0.5, 5, 10, 50, 100, 250 and 500 mg/m² were administered.

The primary objective of the study was to establish the maximum tolerated dose of antibody, and secondary objectives included: (1) demonstrating a reduction in amyloid burden as evidenced by a decrease in affected organomegaly and/or improved organ function; (2) determining the pharmacokinetics of the antibody when given as a single IV infusion (phase 1a) or as a series of weekly IV infusions (phase 1 b); and (3) determining the difference between 250 mg/m² and 500 mg/m² doses.

Key inclusion criteria included being 21 years of age or older, the patient had previously received systemic therapy, the patient did not require plasma cell targeted therapy, and the patient had an Eastern Cooperative Oncology Group (ECOG) performance status of less than or equal to 3.

Key exclusion criteria included intraventricular septum of greater than 2.5 mm, creatine clearance less than 30 cc/min, alkaline phosphatase more than 3 times the institutional upper limit of normal, and bilirubin higher than 3.0 mg/dL.

For the Phase 1a study, dose escalation followed an “up and down design.” Once tolerated, successive patients each received progressively higher doses of antibody, with two patients enrolled at a dose of 500 mg/m². Even the patients receiving the 500 mg/m² dose did not report any dose limiting toxicities. Patients were evaluated at week 0, dosed with antibody at week 1, and then reevaluated at weeks 2, 3, 4, and 8.

For the Phase 1 b study, infusions were given once a week for four weeks starting at 0.5 mg/m². Once tolerated, successive patients each received progressively higher doses of antibody, with six patients enrolled at a dose of 500 mg/m².

Results

Twenty-seven patients were treated with antibody. Twenty-six patients were evaluable for response. Eight patients completed phase 1a and nineteen patients completed treatment in phase 1b. Median age for Phase 1a and 1b was 68. All patients tolerated the given dose of mAb and up to the highest dose level of 500 mg/m² for both phase 1a and 1b. There were no drug-related grade 4 or 5 adverse events (AEs) (grade 5 AEs include, but are not limited to, drug related deaths) or dose-limiting toxicities. Two patients developed a grade 2 rash 3-4 days after infusion. One patient developed the skin rash in in phase 1a (dose level 4) and when he was retreated in phase 1b. A skin biopsy with immunohistochemical staining showed the antibody binding to amyloid fibrils with a concomitant neutrophilic infiltrate. The same patient and another patient developed a similar rash in phase 1b, which further provides clinical and correlative data that the antibody directly binds to light-chain amyloid fibrils. Overall, 63% (5 of 8) of evaluable patients demonstrated organ response after one infusion of the antibody in phase 1a. The median time to response in phase 1a was 4.5 weeks after completing therapy. In phase 1b, 61% (11 of 18) of evaluable patients showed significant organ responses with a median time to response of 1 week after commencing therapy with the tendency of faster response in higher dosages. Further, cardiac response was seen in 67% (8/12) and renal responses were seen in 33% (4/12) of evaluable patients.

The patient characteristics of a subset of the evaluable patients are shown in Table 3 below.

TABLE 3
Phase 1a/b Patient Characteristics

| Characteristic | Median | | | |
|---|---|---|---|---|
| Age (N = 21 patients) | 67 yrs | | | |
| | (Range: 34-77)) | | | |
| Gender | | Male | N = 15 | (68%) |
| | | Female | N = 6 | (32%) |
| Light Chain type | | λ | N = 13 | (52%) |
| | | K | N = 8 | (48%) |
| Revised Mayo Stage | II (Range: I to IV) | | | |
| Organ Involvement (No.) | 2 (Range: 1-4) | Heart | N = 11 | (52%) |
| | | Kidney | N = 11 | (52%) |
| | | Skin/Soft tissue | N = 10 | (48%) |
| | | GI | N = 8 | (38%) |
| | | Nervous system | N = 4 | (19%) |
| | | Liver | N = 3 | (14%) |
| | | Lung | N = 2 | (10%) |
| | | Musculoskeletal | N = 1 | (5%) |
| Best Hematologic Response | | CR | N = 3 | (14%) |
| to Therapy | | | | |
| | | VGPR | N = 15 | (71%) |
| | | PR | N = 2 | (10%) |
| | | NR | N = 1 | (5%) |
| Previous Regimen (No.) | 2 (Range: 1-6) | | | |
| Baseline NT-proBNP (ng/L)^{a} | 2359 (Range: | | | |
| | 894-13,131) | | | |
| Baseline 24 hr Urine Protein | 4998(Range: | | | |
| (mg/24 hr)^{b} | 1078-10,170) | | | |
| Time Since last Exposure to | 6 (Range 1-51) | | | |
| Chemotherapy (mos) | | | | |

^{a}Baseline NT-proBNP in patients with cardiac involvement who were evaluable for response (Baseline NT-proBNP ≥650 pg/ml)
^{b}Baseline 24-hour urine protein in patients with renal involvement who were evaluable for response (Baseline 24-hour urine protein >500 mg/24 h)

At the close of the Phase 1a/b studies, 18 patients had evaluable responses (N=1 had no measurable disease, N=2 did not complete treatment). Twelve of the 18 (67%) showed an improved organ response. Specifically, in the Phase 1a, 63% of patients (5 of 8) with measurable disease burden demonstrated organ response after one infusion of the antibody (2 renal, 2 cardiac, and 1 GI). In the Phase 1b study, 70% of patients (7 of 10) with measurable disease burden showed organ response: 3 of 4 patients who were evaluated for response with cardiac involvement showed cardiac response; 4 of 4 patients who were evaluated for response with renal involvement showed renal response; 1 patient with GI response was evaluated; and 1 patient with soft tissue response showed an improvement of arthritis from ° 3-4° 1.

Cardiac Response

Eight patients were evaluated for cardiac response. Among the metrics that were evaluated were NT-proBNP and NYHA class criteria. The baseline level for all of these patients was 650 pg/ml. Five of the patients (63%) showed a significantly improved response (i.e., a ≥30% decrease in NT-proBNP and/or shifting from NYHA class III to class I), 2 patients remained stable, and only one showed any sign of disease progression.

Renal Response

Eight patients were evaluated for renal response, with proteinuria being the primary metric for determining responsiveness. Six patients (75%) showed a significantly improved response (i.e., a ≥30% decrease in proteinuria or a decrease to <0.5 g/24 hours in the absence of renal progression), and two patients remained stable. No patients showed signs of renal disease progression (>25% worsening in eGFR).

Study Overview of Results

Treatment with antibody was well tolerated and safe. There were no drug related grade 4 or 5 adverse events (AEs) or dose limiting toxicity up to an MTD of 500 mg/m². Moreover, the antibody is clinically efficacious. Most patients saw an early and sustained organ response even as a single infusion or as a weekly infusion for 4 weeks. Improved responses were observed across tissues/organs, including cardiac, renal, GI, skin, and soft tissue responses. Indeed, the antibody safely promotes amyloid resolution in 67% of the patients and leads to improvement in organ function after just a single dose, even in patient with ALX deposits. Patient response to the antibody was rapid and sustained. Indeed, with median response time of 4.5 weeks in the Phase 1a trial and just one week in the Phase 1b trial, the antibody provides a positive response faster than any other known therapeutic targeting amyloid fibrils. The rapid destruction of amyloid fibrils by the antibody can improve organ function and, by extension, significantly improve mortality in patients with this uniformly fatal disease.

### Example 3

### Cardiac Response to Chimeric Fibril-Reactive Monoclonal Antibody in Patients with AL Amyloidosis with Global Longitudinal Strain: Results from a Phase 1B Trial

An open-label phase 1b clinical trial of the antibody described herein was completed with promising results. This study was undertaken to assess the response of myocardial function to mAb administration using global longitudinal strain (GLS).

Nineteen patients with relapse or refractory AL Amyloidosis were enrolled into the trial (age±SD, 63±12; 68% male). Fifty three percent had light chain kappa amyloid and 52% had cardiac involvement as defined by NTpro-BNP level of >650 pg/ml. NTpro-BNP screening and baseline levels of the nineteen patients are shown in Table 9 below. These cardiac patients included 2 patients who were not in the cardiac evaluable primary clinical analysis, due to differences in Screening vs Baseline NT-proBNP values.

The mAb was administered weekly for 4 weeks with sequential doses of 0.5, 5, 10, 50, 100, 250 and 500 mg/m² in a dose-escalation design. Clinical echocardiographic (ECHO) examinations at baseline and 12 weeks post therapy were compared. Several echocardiographic variables including left ventricular ejection fraction (LVEF) (calculated using Simpson's biplane method) and global longitudinal strain (GLS) were obtained. GLS was measured using speckle-tracking (TomTec-Arena 1.2, Germany) and calculated as an average of 4-, 2-, and 3-chamber based measurements. Paired student's t-test was used to compare echocardiographic variables at baseline and 12 weeks after therapy with mAb. The analysis of the echocardiogram parameters are demonstrated in Table 4 below.

TABLE 4
The analysis of the echocardiographic parameters

| | | | | | P- |
|---|---|---|---|---|---|
| | Screening | | Week 12 | | value |
| Number Patients | 10 | | 10 | | |
| lvidd_cm (mean (sd)) | 4.38 | (0.93) | 4.32 | (0.91) | 0.319 |
| ivs_cm (mean (sd)) | 1.29 | (0.22) | 1.21 | (0.18) | 0.119 |
| pwt_cm (mean (sd)) | 1.12 | (0.27) | 1.14 | (0.25) | 0.217 |
| lved_mass_g (mean (sd)) | 187.86 | (43.37) | 179.30 | (42.87) | 0.197 |
| ef_percent (mean (sd)) | 51.95 | (9.92) | 52.28 | (11.59) | 0.856 |
| mcf (mean (sd)) | 0.37 | (0.11) | 0.38 | (0.13) | 0.626 |
| mv_e_m_s (mean (sd)) | 8.53 | (24.18) | 0.94 | (0.37) | 0.347 |
| mv_a_m_s (mean (sd)) | 13.45 | (34.05) | 0.72 | (0.23) | NA |
| gls_percent (mean (sd)) | 15.68 | (4.14) | 17.37 | (3.53) | 0.004 |

Novel anti-plasma cell therapies continue to emerge with improvements in hematologic responses. Organ response even after hematologic response is established is still unpredictable. Persistent organ dysfunction irrespective of hematologic response remains an issue. There remains a need for therapies that target amyloid fibril removal to prevent and reverse organ damage.

While there was neither significant change between LVEF (56.2±8.6% vs. 56.2±9.5%, p=0.985) nor GLS (−19.04+−5.11% vs. −19.73±−4.1%, p=0.119) from baseline to 12 week examinations for the overall cohort, patients with cardiac involvement demonstrated an improvement in GLS (−15.58±−4.14% pre and −17.37±−3.53% post, p=0.004). An exemplary echocardiogram of a patient with cardiac involvement before treatment and at week 12 post treatment with the antibodyhad a baseline level of NT-proBNP of 2549 pg/mL and a GLS value of −9.58 before treatment. After 12 weeks of mAb treatment, the patient exhibited reduction in GLS to −13.39, and a reduction in NTproBNP to 1485 pg/mL. Subgroup analysis showed an improvement in GLS in patients with lambda amyloid cardiac involvement (−14.3±−4.38% pre and −16.17±−3.74% post, p=0.02) and a trend in improvement with kappa amyloid cardiac involvement (−16.60±−4.10% pre and −18.16±−3.48% post, p=0.07). Furthermore, the Cardiac Evaluable Population as defined in the clinical analysis of the study (using the Baseline NT-proBNP values rather than the Screening Values) also resulted in a statistically significant decrease in GLS % (p-value 0.0163), with a numerically similar decrease (−1.71) as the analysis provided by the ECHO group (−1.69). Table 5 below shows the analysis of decrease in the GLS in cardiac patients versus cardiac evaluable patients and non-cardiac patients.

TABLE 5
Comparison of Cardiac Patients per Screening vs Baseline
Evaluable Definitions and Non Cardiac Patients.

| | Number Patients | Baseline | Follow-up | P-value |
|---|---|---|---|---|
| GLS % Mean | 10(Cardiac Patients | −15.68 (4.14) | −17.37 (3.53) | 0.004 |
| (Std Dev) | per Screening NT-proBNP) | | | |
| | 8(Cardiac Evaluable per Baseline: | −14.95 (4.32) | −16.66 (3.55) | 0.0163 |
| | NT- proBNP) | | | |
| | 9 (Non-Cardiac Patients per | 22.77 (3.12) | −22.36 (3.02) | 0.4829 |
| | Semening NT-proBNP) | | | |

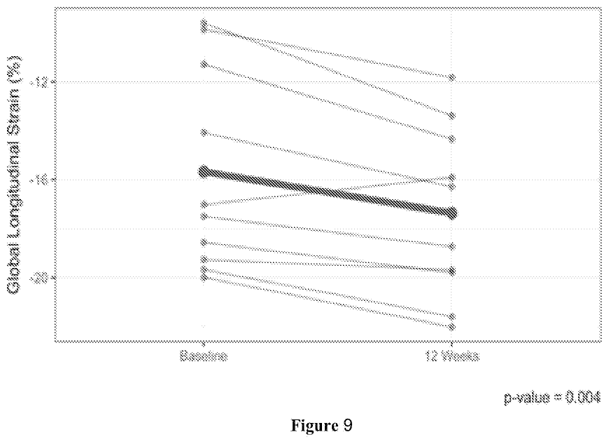

In conclusion, this trial shows a significant improvement in GLS after exposure to the disclosed antibody, an anti-fibril specific mAb, in subjects with AL amyloid cardiac involvement. Phase 1 administration of the disclosed antibody resulted in improvement in GLS in cardiac patients when observed over 12 weeks as illustrated in FIG. 9. 9 out of 10 patients with cardiac involvement improved on GLS %. The probability that 9 or more patients improving, under the null hypothesis that there is no drug effect, is ˜0.0107, suggesting that observing 9 of 10 patients improving is a highly unlikely outcome unless the drug truly is effective.

### Example 4

### Organ Response to Chimeric Fibril-Reactive Monoclonal Antibody in Patients Who are not Controlled Hematologically

A patient that had received 6 chemotherapy treatments and had achieved a hematologic partial response on those treatments with no organ response was administered the amyloid fibril-reactive monoclonal antibody (mAb) described herein. For three consecutive periods post-dose, there was consistent reduction in NT-proBNP after receiving the antibody, and the patient achieved an organ response. But when taken off of antibody, free light chains increased, and the patient condition worsened. Organ progression was then seen after completion of the trial. The response pattern of this patient led the investigators to conclude that organ response was due to the antibody treatment and independent of chemotherapy induced hematologic response.

### Example 5

### Results of a Phase 2 Study of Tolerability of the Antibody in AL Amyloidosis Patients Receiving Concomitant Cyclophosphamide-Bortezomib-Dexamethasone (Cybord)

The antibody of the present disclosure was studied as a monotherapy in an open label dose escalation Phase 1 study where it was shown to be safe, tolerable up to 500 mg/m² and associated with an early organ response. No dose-limiting toxicities (DLTs) were seen and the PK profile appeared not to reach linearity. It was hypothesized that the disclosed antibody will modify the disease course of AL amyloidosis by facilitating the removal of amyloid deposited in tissues.

The goal of the multicenter, open-label, sequential cohort, dose-selection study (3+3) of the antibody in Mayo Stage I, II and IIIa AL amyloidosis patients was to define the safety and tolerability of the antibody in combination with cyclophosphamide-bortezomib-dexamethasone (CyBorD) as the standard of care (SOC) plasma cell dyscrasia (PCD) therapy during a 27 day treatment period, and to determine the recommended dose for the subsequent Phase 3 studies.

The study was divided into two parts with the following objectives: Part A defined the safety and tolerability of the disclosed antibody in combination with SoC CyBorD and determined the recommended Phase 3 (RP3D) of the disclosed antibody; Part B evaluated the safety and tolerability of the disclosed antibody in combination with Soc CyBorD and daratumumab.

Patient had measurable hematologic disease, as defined by at least one of:
FLC>5 mg/dL with abnormal ratio or
Serum protein electrophoresis (SPEP) m-spike>0.5 g/dL

Patients were seen in the clinic every week for 4 weeks to receive a 2 hrs IV infusion of the antibody, and for safety and tolerability assessments. Subsequently, patients have been receiving antibody infusions approximately every other week, as a continuing maintenance dose.

Actual dose was determined by patient body surface area in meters squared. When administered on the same day, the antibody was administered first before CyBorD chemotherapy.

Part A

Part A of the study employed a 3+3 dose escalation design, starting with a 500 mg/m² dose. Patients were followed for dose-limiting toxicities (DLTs). The DLT observation period for the first cohort was through 14 days following the first infusion. For subsequent cohorts, this was through 27 days. Enrollment into a new cohort with the next higher dose of the antibody did not begin until the DLT observation period had been completed for the last patient enrolled in the previous cohort, with further dosing cohorts at 750 and 1000 mg/m². At least 3 patients were enrolled in each dose cohort with cohort 1 (n=4) at 500 mg/m², cohort 2 (n=3) at 750 mg/m², and cohort 3 (n=6) at 1000 mg/m² (see Table 6). DLT observation period was weekly for four weeks. Safety assessment included vital signs, physical examinations, electrocardiograms (ECGs), immunogenicity assessments (ADA), clinical laboratory parameters (hematology, serum chemistry, urine analysis), and TEAEs. The pharmacokinetic (PK) profile of the antibody was also assessed.

As illustrated in Table 6, the 13 patients averaged 65.2 years (range 47.6 to 79.6 years). The majority were male (76.9%), white (84.6%), and non-Hispanic (100%). Mayo staging I (7.7%), II (69.2%), and III (23.1%) reflected a wide range of disease severity in the patients enrolled. All patients were treated successfully through their 4th dose, the highest (1000 mg/m²) cohort enrolling 6 patients. No DLT was observed, and the antibody was well-tolerated by all patients. The most common TEAEs were diarrhea and nausea (each 30.8%) (Table 7). Dose-normalized PK concentrations were best described by a linear two-compartment model, with terminal half-life of 28 days.

TABLE 6

| | | | | | | | Antibody 1000 | | | |
|---|---|---|---|---|---|---|---|---|---|---|
| | Antibody 500 | | Antibody 750 | | Antibody 1000 | | mg/m² + | | | |
| | mg/m² + | | mg/m² + | | mg/m² + | | CyBorD + Dara | | | |
| | CyBorD | | CyBorD | | CyBorD | | (interim data) | | All Patients | |
| | N = 4 | | N = 3 | | N = 6 | | N = 2 | | N = 15 | |
| Median age, years | 55 | (49 to 62) | 7-(48 to 70) | | 75 | (62 to 80) | 63 | (63 to 64) | 64 | (48 to 80) |
| (range) | | | | | | | | | | |
| Sex, n (% male) | 4 | (100%) | 2 | (66.7%) | 4 | (66.7%) | 2 | (100%) | 12 | (80%) |
| Number Previous | 3-4 | | 0-4 | | 0-4 | | Not yet available | | 0-4 | |
| PCD treatments | | | | | | | | | | |
| range | | | | | | | | | | |
| Time since | 46 | (19 to 163) | 13 | (3 to 23) | 12 | (3 to 23) | 3 | (2 to 3) | 13 | (2 to 163) |
| Diagnosis | | | | | | | | | | |
| (months), median | | | | | | | | | | |
| (range) | | | | | | | | | | |
| Mayo Stage n (%) | | | | | | | | | | |
| I | 1 | (25) | 0 | | 0 | | 0 | | 1 | (6.7) |
| II | 2 | (50) | 2 | (66.7) | 5 | (83.3) | 2 | (100) | 11 | (73.3) |
| III | 1 | (25) | 1 | (33.3) | 1 | (16.7) | 0 | | 3 | (20) |
| AL amyloidosis | 2 | (50) | 3 | (100) | 3 | (50) | 2 | (100) | 10 | (66.7) |
| in Heart n | | | | | | | | | | |
| (NTproBNP >650 | | | | | | | | | | |
| pg/mL, %) | | | | | | | | | | |
| NT-ProBNP | 928 | (50 to 3712) | 752 | (612 to 1002) | 2926 | (545 to 18696) | 1799 | (823 to 2774) | 1002 | (50 to 18696) |
| (pg/mL) median | | | | | | | | | | |
| (range) | | | | | | | | | | |
| cTnT (ng/mL) | 0.034 | (0.010 to 0.041) | 0.018 | (0.010 to 0.047) | 0.012 | (0.010 to 0.106) | 0.019 | (0.013 to 0.024) | 0.016 | (0.010 to 0.106) |
| median (range) | | | | | | | | | | |
| Baseline, eGFR, | 45 | (34 to 54) | 60 | (44 to 60) | 33 | (22 to 60) | 101 | (84 to 118) | 47 | (22 to 118) |

median (range)

This study demonstrated that the presently described antibody at doses up to 1000 mg/m², given with CyBorD, was well-tolerated in the AL amyloidosis population. Two Phase 3 efficacy/safety studies, one enrolling Mayo Stage IIIa and the other Stage IIIb patients, have been initiated, and based on the findings from this Phase 2 study, the 1000 mg/m² dose of the antibody is being used for treatment in those studies.

TABLE 7

| | Cohort 1: | | Cohort 2: | | Cohort 3: | | | |
|---|---|---|---|---|---|---|---|---|
| | 500 mg/m² | | 750 mg/m² | | 1000 mg/m² | | Total | |
| MedDRA Preferred Term | N = 4 | | N = 3 | | N = 6 | | N = 13 | |
| At least one TEAE | 4 | (100.) | 3 | (100.0) | 6 | (100.0) | 13 | (100.0) |
| At least one TEAE considered to be | 2 | (50.0) | 1 | (33.3) | 1 | (16.7) | 4 | (30.8) |
| at least possibly related ¹ | | | | | | | | |
| At least one Grade 3 or higher TEAE | 0 | | 0 | | 1 | (16.7) | 1 | (7.7) |
| At least one treatment-emergent SAE | 0 | | 0 | | 2 | (33.3) | 2 | (15.4) |
| TEAEs leading to study | 0 | | 0 | | 0 | | 0 | |
| discontinuation | | | | | | | | |
| TEAEs leading to death | 0 | | 0 | | 0 | | 0 | |
| At least one DLT TEAE | 0 | | 0 | | 0 | | 0 | |
| Most Common TEAEs (>10% in | | | | | | | | |
| Total Column) | | | | | | | | |
| Diarrhea | 0 | | 1 | (33.3) | 3 | (50.0) | 4 | (30.8) |
| Nausea | 2 | (50.0) | 1 | (33.3) | 1 | (16.7) | 4 | (30.8) |

| Insomnia | 2 | (50.0) | 0 | 1 | (16.7) | 3 | (23.1) |
|---|---|---|---|---|---|---|---|
| Fatigue | 1 | (25.0) | 0 | 1 | (16.7) | 2 | (15.4) |
| Hyperkalemia | 1 | (25.0) | 0 | 1 | (16.7) | 2 | (15.4) |
| Anaemia | 0 | | 0 | 2 | (33.3) | 2 05.4) | |
| Headache | 1 | (25.0) | 1 (33.3) | 0 | | 2 | (15.4) |
| Rash | 1 | (25.0) | 0 | 1 | (16.7) | 2 | (15.4) |

Abbreviations: TEAE = treatment-emergent adverse event. A TEAE was defined as an adverse event occurring during or after study dmg administration. SAE = Serious TEAE. DLT = Dose-limiting-toxicty.
¹ At least possibly related TEAEs were defined as TEAEs with relationship of probably, possibly, or definitely related.
Note:
Patients experiencing more than 1 TEAE were only counted once under the

Part B

In Part B of the study, patients (N=2) were treated with the CyBorD in combination with daratumumab (dara) and the disclosed antibody at 1000 mg/m². DLT observation period was weekly for four weeks followed by every other week.

Bortezomib 1.3 mg/m² subcutaneous, cyclophosphamide 300 mg/m² (dose capped at 500 mg) IV and dexamethasone 20 mg IV (CyBorD) were given weekly, day 1, 8, 15 of a 35 day cycle in the first cycle to align treatments with the disclosed antibody and then weekly day 1, 8, 15 of a 28 day cycle for up to 6 total cycles. The disclosed antibody was continued every other week after the conclusion of CyBorD. No dose adjustments of the disclosed antibody could be made during the dose escalation phase. Dose adjustments in CyBorD due to toxicity were made at the discretion of the treating physician. If the recommended phase 3 dose was proven to be 1000 mg/m², patients at lower dose levels were permitted to increase their dose to 1000 mg/m².

Key inclusion and exclusion criteria was as follows: newly diagnosed and relapsed patients were permitted; patients with multiple myeloma and AL amyloidosis were excluded; and Mayo stage IIIb were excluded.

Safety and Dosing Summary

The disclosed antibody was administered between 40 and 276 days (data cut 11/01/2020). The disclosed antibody was shown to be safe and well tolerated in combination with CyBorD with 1000 mg/m² established as recommended Phase 3 dose for CARES ongoing trials. No dose limiting toxicity or treatment related discontinuation was observed. 2 patients escalated to 1000 mg/m² were dose reduced to 750 mg/m² due to diarrhea and vomiting, respectively. Treatment is ongoing with 40% of patients having at least 15 infusions of the disclosed antibody. No infusion reactions were seen with premedication with 1 gram oral acetaminophen and 25 mg oral diphenhydramine.

The most common TEAEs at least possibly related to study treatment are shown in Table 10. The most common TEAEs irrespective of relationship to the disclosed antibody were diarrhea and nausea, similar to Phase 1. The two patients who had dose reductions from 1000 mg/m² to 750 mg/m² (for diarrhea and vomiting, respectively), showed the TEAEs subsequently resolving.

TABLE 8

| | | | | Antibody | |
|---|---|---|---|---|---|
| | Antibody | Antibody | Antibody | 1000 mg/m² + | |
| | 500 mg/m² + | 750 mg/m² + | 1000 mg/m² + | CyBorD + | |
| | CyBorD | CyBorD | CyBorD | Dara | All Patients |
| CTCAE Preferred Term | N = 4 | N = 3 | N = 6 | N = 2 | N = 15 |
| At least one at least | 2 (50.0) | 1 (33.3) | 2 (33.3) | 0 | 5 (33.3) |
| Possibly Related TEAE | | | | | |
| Rash | 1 (25.0) | 1 (33.3) | 1 (16.7) | 0 | 3 (20.0) |
| Diarrhea | 1 (25.0) | 0 | 0 | 0 | 1 (6.7) |
| Nausea | 1 (25.0) | 0 | 0 | 0 | 1 (6.7) |
| Dyspnea | 1 (25.0) | 0 | 1 (16.7) | 0 | 1 (6.7) |
| Anemia | 0 | 0 | 1 (16.7) | 0 | 1 (6.7) |

Abbreviations: TEAE = treatment-emergent adverse event. A TEAE was defined as an adverse event occuring during or after study dmg administration. This table includes all TEAEs judged by the investigator to be at least Possibly

The most common TEAs at least possibly related to study treatment are shown in Table 8.

### Example 6

### Preliminary Results of the Antibody Phase 2 Pharmacokinetic Analysis

The objectives of the preliminary phase 2 pharmacokinetic (PK) data analysis were (1) to assess dose-proportionality in PK exposures from 500 to 1000 mg/m²; (2) to assess the lowest dose/smallest dose number to reach a target C_{trough} of 130 μg/mL; and (3) to assess Phase 3 dose and regimen recommendation with partial Phase 2 PK data.

TABLE 9
Comparison of the Phase 2 vs. Phase 1b study

| | Phase 1b | Phase 2 |
|---|---|---|
| Patient | Patients with AL amyloidosis who | Similar |
| | were previoulsy treated | |
| Antibody Material | Not optimal | New higher producing cell line |
| | | New manufacturer with platform process for |
| | | scalability |
| | | Removal of any animal components |
| | | Comparable with Phase 1 material by |
| | | analytical and animal exposure (PK) |
| | | assessments |
| CyBorD | Some had pre-dose chemo | Added CyBorD: chemotherapy to be used in |
| | | Phase 3 dosing |
| PK Assay | Non-validated research level | |
| | ELISA at KCAS: LLOQ = 0.5 μg/ml | |

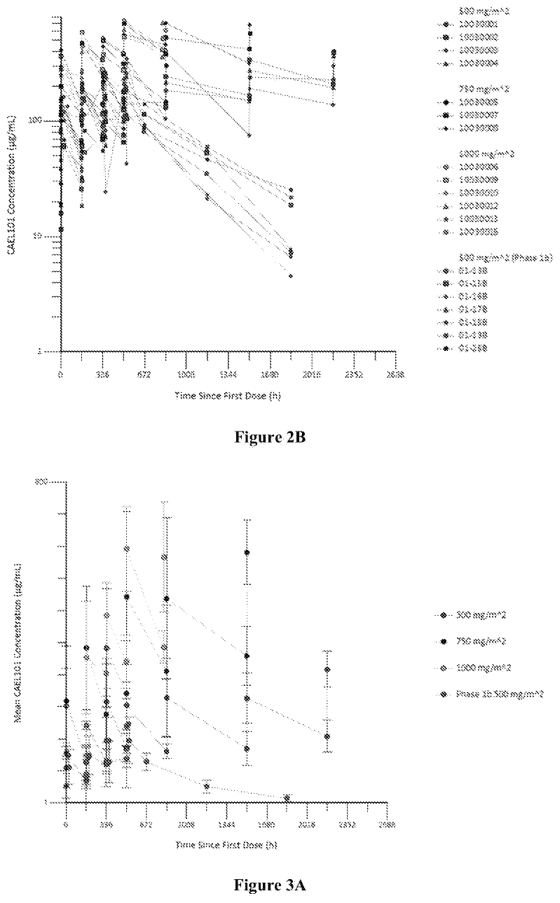

Individual PK of the antibody of the present disclosure was evaluated over time and compared to the data of the Phase 1b study. As illustrated in FIGS. 2A and 2B, in the phase 2 study, 500, 750, and 1000 mg/m² doses had complete peak (highest concentration of the antibody in the patient's bloodstream) and trough (lowest concentration in the patient's bloodstream before administration) data for dose 6, 4, and 3, respectively), and shown an increase in PK exposure from 500 to 1000 mg/m². This is in contrast with the data from the phase 1b study, where concentrations were lower and variability higher.

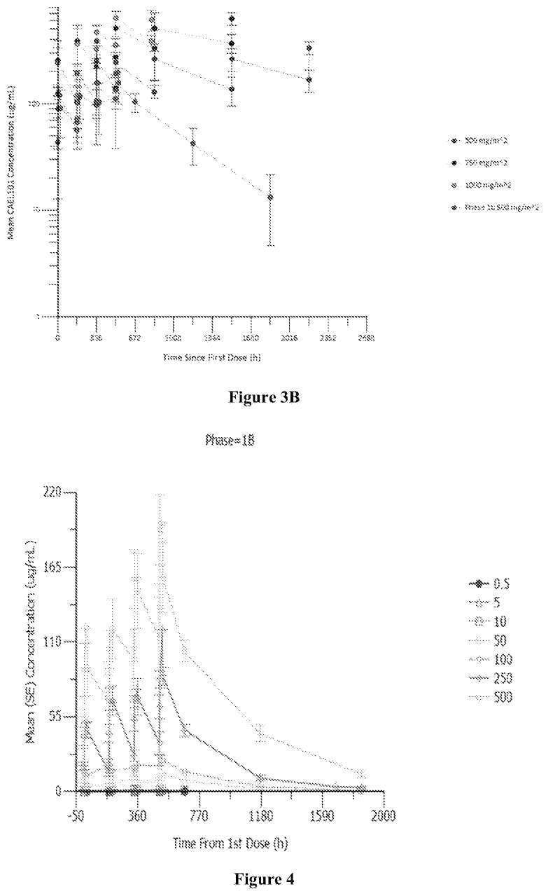

As further shown in FIGS. 3A and 3B study of mean PK of the antibody was evaluated over time and compared to the data of the Phase 1b study. In the phase 2, 500, 750, and 1000 mg/m² doses had complete peak and trough data for dose 6, 4, and 3, respectively; and shown an increase in PK exposure with dose from 500 to 1000 mg/m². This is in contrast with the data from the phase 1b study, where concentrations were lower and variability higher, especially after doses 2 to 4.

The study of the antibody peak exposure Cₘₐₓ (μg/mL) by dose was further evaluated. As shown in Table 10, at 500 mg/m² overall the peak exposures were comparable between Phase 2 and Phase 1b studies. There were overlapping Cₘₐₓ after doses 1 and 4, and similar dose 3/dose 1 accumulation ratio of ˜1.43 to 2-fold. The phase 2 had approximately 50% higher Cₘₐₓ after doses 2 and 3 as compared to the Phase 1b.

In this phase 2 study, there was low to moderate variability. At 1000 mg/m², the last 3 data point added variability (lower PK; slightly more severe disease status). At 750 mg/m², the highest % CV was due to data point 1003-0005, a subject with Cardiac, Mayo Stage 2, who was switched to daratumumab.

TABLE 10

| | Dose 1 | | | Dose 2 | | | Dose 3 | | |
|---|---|---|---|---|---|---|---|---|---|
| | 500 | 750 | 1000 | 500 | 750 | 1000 | 500 | 750 | 1000 |
| Cₘₐₓ | mg | mg | mg | mg | mg | mg | mg | mg | mg |
| N | 4 | 3 | 6 | 4 | 3 | 6 | 4 | 3 | 3 |
| Mean | 126 | 255 | 244 | 194 | 387 | 363 | 253 | 387 | 468 |
| SD | 16.7 | 136.5 | 92.8 | 9.8 | 153.3 | 140.5 | 14.3 | 147.0 | 81.7 |
| Min | 113 | 154 | 134 | 182 | 210 | 210 | 236 | 220 | 374 |
| Median | 120 | 200 | 267 | 196 | 472 | 339 | 254 | 444 | 505 |
| Max | 150 | 410 | 367 | 204 | 479 | 586 | 267 | 497 | 524 |
| CV % | 13% | 54% | 38% | 5% | 40% | 39% | 6% | 38% | 17% |
| GeoMean | 125.0 | 232.9 | 227.1 | 194.1 | 362.1 | 341.5 | 252.4 | 364.8 | 462.6 |
| GeoCV % | 12.7% | 54.1% | 44.4% | 5.1% | 49.9% | 39.8% | 5.7% | 46.4% | 18.7% |

| | Dose 4 | | | Dose 5 | | | Dose 6 | | |
|---|---|---|---|---|---|---|---|---|---|
| | 500 | 750 | 1000 | 500 | 750 | 1000 | 500 | 750 | 1000 |
| Cₘₐₓ | mg | mg | mg | mg | mg | mg | mg | mg | mg |
| N | 4 | 3 | 3 | 4 | 3 | 3 | 4 | 2 | 0 |
| Mean | 244 | 514 | 633 | 263 | 510 | 613 | 262 | | |
| SD | 102.3 | 212.1 | 105.3 | 98.1 | 200.5 | 96.0 | 62.5 | | |
| Min | 138 | 283 | 533 | 184 | 302 | 517 | 192 | | |
| Median | 245 | 559 | 624 | 231 | 526 | 613 | 258 | | |
| Max | 349 | 700 | 743 | 405 | 702 | 709 | 340 | | |
| CV % | 42% | 41% | 17% | 37% | 39% | 16% | 24% | | |
| GeoMean | 227.1 | 480.2 | 627.5 | 250.6 | 481.3 | 607.9 | 256.2 | | |
| GeoCV % | 46.9% | 49.9% | 16.7% | 35.0% | 44.9% | 15.9% | 24.4% | | |

As illustrated in FIG. 4, antibody systemic exposure was found slightly above dose proportional across dose range from 0.5 to 500 mg/m² (phase 1b study). Mean T_{1/2} were 10 days and 16 days respectively for 250 and 500 mg/m² in the phase 1b study, and the model with QuantPharm showed T1/2 closer to 24 days. The Cₘᵢₙ was set at the average below 3 numbers, which was 130 ug/mL. NTproBNP started to increase 1 week post last dose (880 hrs) when Cₘᵢₙ for 500 mg/m² was dropping ˜100 ug/mL (N=7 pt). Other biomarkers were highly variable.

Half maximal effective concentration (EC50) for highest binding amyloid (in vitro) was ˜157 ug/mL, Michaelis-Menton (MM) EC 90 (calculated) was ˜36.5 ug/mL (95 CI 5-134 ug/mL).

Modeling and simulation of the Phase 1a/1b indicated that the minimum predicted steady-state C_{minSS} (among patients from the analysis data set) achieved this level following: 250 mg/m² QW, 750 mg/m² Q2W, or 1000 mg/m² Q3W dosing regimens. With Q4W dosing, even 1000 mg/m² dose achieved only 82.7% target occupancy at C_{minSS} for a patient with the lowest exposure.

The study antibody minimum exposure Cₘᵢₙ (C_{trough}, μg/mL) was evaluated by dose. As shown in Table 11, at 500 mg/m² overall the trough exposures overlapped between Phase 2 and Phase 1b, with Phase 2 trending higher. Between Phase 2 and 1b dose 3/dose 1 accumulation ratio was 2.3 vs. 4.4-fold. In this phase 2 low to moderate variability was observed.

Minimum C_{trough} for 90% receptor occupancy was achieved at >130 μg/mL, after 2nd weekly dose under 1000 mg/m²; 3rd weekly dose under 750 mg/m²; 6th weekly dose under 500 mg/m². The 130 μg/mL target was based on the phase 1a/1b study and may likely change with new Phase 2 data.

TABLE 11

| | Dose 1 | | | Dose 2 | | | Dose 3 | | |
|---|---|---|---|---|---|---|---|---|---|
| | 500 | 750 | 1000 | 500 | 750 | 1000 | 500 | 750 | 1000 |
| Cₘᵢₙ | mg | mg | mg | mg | mg | mg | mg | mg | mg |
| N | 4 | 3 | 6 | 4 | 3 | 3 | 4 | 3 | 3 |
| Mean | 57 | 102 | 119 | 104 | 222 | 324 | 139 | 274 | 353 |
| SD | 8.5 | 43.3 | 75.8 | 31.2 | 124.5 | 37.6 | 35.1 | 130.7 | 66.2 |
| Min | 48 | 55 | 26 | 73 | 91 | 281 | 108 | 127 | 277 |
| Median | 55 | 111 | 113 | 101 | 238 | 340 | 135 | 316 | 388 |
| Max | 68 | 140 | 241 | 141 | 338 | 351 | 179 | 378 | 395 |
| CV % | 15% | 42% | 64% | 30% | 56% | 12% | 25% | 48% | 19% |
| GeoMean | 56.0 | 94.8 | 95.9 | 100.5 | 193.8 | 322.5 | 135.7 | 247.5 | 348.8 |
| GeoCV % | 14.7% | 51.9% | 92.7% | 30.9% | 77.0% | 12.1% | 25.7% | 63.9% | 20.2% |

| | Dose 4 | | | Dose 5 | | | Dose 6 | | |
|---|---|---|---|---|---|---|---|---|---|
| | 500 | 750 | 1000 | 500 | 750 | 1000 | 500 | 750 | 1000 |
| Cₘᵢₙ | mg | mg | mg | mg | mg | mg | mg | mg | mg |
| N | 4 | 3 | 3 | 4 | 2 | 0 | 4 | 2 | 0 |
| Mean | 129 | 330 | 396 | 136 | | 193 | | | |
| SD | 17.9 | 163.2 | 31.3 | 41.3 | | 38.4 | | | |
| Min | 105 | 147 | 360 | 76 | | 139 | | | |
| Median | 132 | 381 | 411 | 152 | | 203 | | | |
| Max | 148 | 461 | 417 | 167 | | 228 | | | |
| CV % | 14% | 49% | 8% | 30% | | 20% | | | |
| GeoMean | 128.3 | 295.6 | 395.1 | 130.4 | | 189.8 | | | |
| GeoCV % | 14.5% | 67.4% | 8.1% | 38.0% | | 22.0% | | | |

The study antibody AUCτ (μg×hr/mL) was evaluated by dose. As shown in Table 14, at 500 mg/m² overall the cumulative exposures were comparable between Phase 2 and Phase 1b studies. There was overlapping AUCτ values after doses 1 and 4. In the phase 2 study, dose 3/dose 1 accumulation ratio was 3.3 to 4 vs. 2-fold in the phase 1b.

In this phase 2 study, there was approximately 100% higher AUCτ after doses 2 and 3 as compared to the phase 1b study; and variability was low to moderate.

TABLE 12

| | Dose 1 | | | Dose 2 | | | Dose 3 | | | Dose 4 | | |
|---|---|---|---|---|---|---|---|---|---|---|---|---|
| AUC_{τ} | 500 mg | 750 mg | 1000 mg | 500 mg | 750 mg | 1000 mg | 500 mg | 750 mg | 1000 mg | 500 mg | 750 mg | 1000 mg |
| N | 4 | 3 | 6 | 4 | 3 | 3 | 4 | 3 | 3 | 4 | 3 | 2 |
| Mean | 15240 | 29879 | 30335 | 25651 | 51159 | 67003 | 32082 | 55333 | 68947 | 62061 | 141780 | |
| SD | 1439 | 14672 | 12592 | 1508 | 22865 | 5049 | 4399 | 23269 | 12331 | 14232 | 60367 | |
| Min | 13773 | 17545 | 13430 | 24286 | 25133 | 62988 | 28302 | 29046 | 54721 | 48016 | 72328 | |
| Median | 14987 | 25986 | 34455 | 25260 | 60324 | 65350 | 31281 | 63660 | 75535 | 62622 | 171357 | |
| Max | 17212 | 46105 | 43881 | 27800 | 68019 | 72671 | 37462 | 73292 | 76585 | 74983 | 181655 | |
| CV % | 9% | 49% | 42% | 6% | 45% | 8% | 14% | 42% | 18% | 23% | 43% | |
| GeoMean | 15190.0 | 27598.5 | 27688.7 | 25618.8 | 46894.7 | 66878.4 | 31859.5 | 51365.2 | 68152.4 | 60818.7 | 131064.9 | |
| GeoCV % | 9% | 52% | 53% | 6% | 59% | 7% | 14% | 53% | 19% | 24% | 55% | |

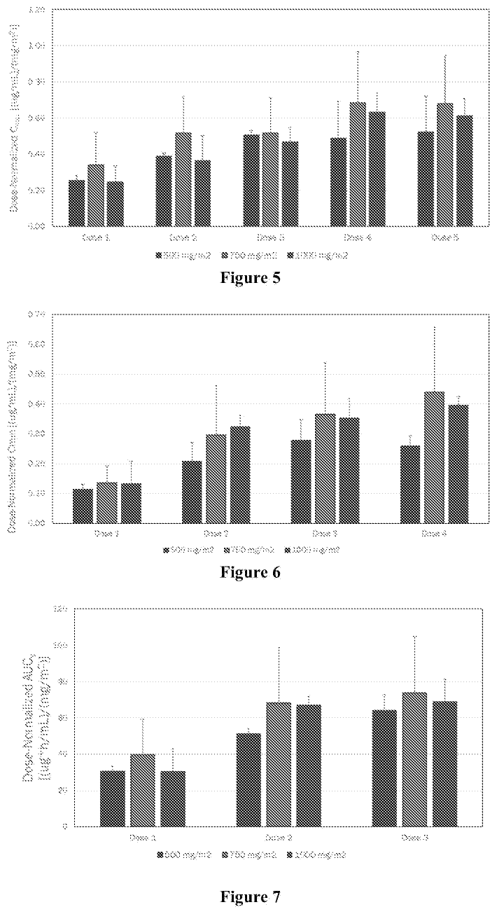

Dose proportionality assessment for Cₘₐₓ/dose was evaluated. As illustrated in FIG. 5 and Table 13, in this phase 2 study, Cₘₐₓ increased approximately dose proportionally from 500 to 1000 mg/m². This was especially the case after dose 3, which indicated saturation of target mediated drug disposition (TMDD) at 750 mg/m².

TABLE 13
Regression Lines

| Dose (N) | m |
|---|---|
| 1 (4, 3, 6) | −0.00002 |
| 2 (4, 3, 6) | −0.00005 |
| 3 (4, 3, 3) | −0.00008 |
| 4 (4, 3, 3) | 0.00030 |
| 5 (4, 3, 3) | 0.00020 |

Further, dose proportionality assessment for Cₘᵢₙ/dose was evaluated. As illustrated in FIG. 6 and Table 14, in this phase 2 study, Cₘᵢₙ increased approximately dose proportionally from 500 to 1000 mg/m². This was especially the case after dose 1, which indicated saturation of target mediated drug disposition (TMDD) at 750 mg/m².

TABLE 14
Regression Lines

| Dose (N) | m |
|---|---|
| 1 (4, 3, 6) | −0.00004 |
| 2 (4, 3, 3) | 0.0002 |
| 3 (4, 3, 3) | 0.0002 |
| 4 (4, 3, 3) | 0.0003 |

Additionally, dose proportionality assessment for AUCτ/dose was evaluated. As illustrated in FIG. 7 and Table 15, AUCτ increased approximately dose proportionally from 750 to 1000 mg/m², indicating saturation of target mediated drug disposition (TMDD) at 750 mg/m².

TABLE 15
Regression Lines

| Dose (N) | m |
|---|---|
| 1 (4, 3, 6) | −0.00030 |
| 2 (4, 3, 3) | 0.03140 |
| 3 (4, 3, 3) | 0.00960 |

In conclusion, the preliminary data of the PK study of the presently described antibody shown that PK exposures increased with increasing doses over the studied dose range of 500 to 1000 mg/m² and that TMDD led to >dose-proportional increase in PK exposures between 500 and 750 mg/m² and approximately dose-proportional increase between 750 and 1000 mg/m². The information supported a dosing with 1000 mg/m², given the maximum tolerated dose with lack of safety concerns, and that all subjects reached >desired Cₘᵢₙ after 2^{nd} dose (immediate, complete, and sustained target saturation). Therefore, this study revealed a P3D (Recommended Phase 3 dose) defined in the Phase 3 study protocols as 4 QW loading dose followed by Q2W maintenance; dose level 1000 mg/m².

The complete gathering of PK and PD data in this Phase 2 at the higher dose levels, the update of PK/PD model integrating Phases 1 and 2 data will be used to refine the Phase 3 optimal dosing regimen and might establish a new target C_{trough}.

While there were limitations of differences in the BA assay for PK, test material, and presence vs. absence of CyBorD (some patients in Phase 1b were treated with chemotherapy before study), antibody exposures following the first dose (Dose 1) overlapped between the two studies but were ˜30-100% higher after subsequent doses for the Phase 2 study.

### Example 7

### Antibody Formulation and Dosage

Antibody Formulation:

The formulation of the antibody for administration to subject was defined as containing:
30 mg/ml antibody, 10 ml per vial (=300 mg antibody),
25 mM Sodium Acetate,
50 mM Sodium Chloride,
1% Mannitol, and
0.01-0.05% Polysorbate 80.

The formulation was kept at pH 5.5

Dose Coverage Evaluation:

The study of population pharmacokinetics has shown no correlation with body surface area (BSA) or Weight. The best dose by BSA was determined to be 1000 mg/m², with a second-best option at 750 mg/m², which might want be used as a step down).

The coverage for all doses between 250-1375 mg/m² revealed that the best dose by weight evaluated at 25 mg/kg; the second-best option being 18.75 mg/kg.

The coverage for all doses between 6.25-31.25 mg/kg revealed that the target-mediated drug disposition (TMDD, the phenomenon in which a drug binds with high affinity to its pharmacological target site) occurred at 750 mg/m² and above; which is a critical observation for appropriated dosing across all patient amyloid subtypes and severity levels.

Surprisingly, all patient subtypes and severities had similar PK exposure profiles where it is expected to see potentially difference in clearance and saturation exposures. Low/medium patient variability was observed both with an antibody dose along and in combination with plasma cell directed therapy (PCD).

Fixed Dose Coverage:

For fixed doses by weight, patients were separated in 3 dose groups receiving 25 mg/kg:
40-70 kgs-1750 mg per dose
71 kg-100 kg-2500 mg/dose
>100 kg-2750 mg/dose

For fixed dose by BSA, patients were separated 3 dose groups:
1.15-1.70 BSA—fixed dose of 1700 mg per dose
1.71-2.40 BSA—fixed dose of 2400 mg per dose
>2.41 BSA—fixed dose of 2750 mg per dose

BSA was calculated by multiple methods, as illustrated in Table 13.

TABLE 16

| Height | Weight (lb) | d&d | G&G | Haycock | Mosteller | | | |
|---|---|---|---|---|---|---|---|---|
| 4′6″ | 77 | 137 | 35 | 1.15 | 1.17 | 1.15 | 1.15 | |
| 5′0″ | 113 | 152 | 50 | 1.45 | 1.47 | 1.46 | 1.45 | |
| 6′2″ | 165 | 188 | 75 | 2 | 1.98 | 1.97 | 1.98 | |
| 5′5″ | 250 | 165 | 113 | 2.17 | 2.31 | 2.33 | 2.28 | |
| 5′9″ | 264 | 178 | 120 | 2.32 | 2.45 | 2.47 | 2.42 | |
| 5′4″ | 170 (average) | | 163 | 1.83 | 1.83 | 1.89 | 1.89 | 1.87 |
| 5′9″ | 200 (average) | | 175 | 2.07 | 2.07 | 2.12 | 2.13 | 2.10 |

Loading Dose Regimen:

C_{trough} levels covered 30 ug/mL-400 ug/mL.

It was determined by binding numbers from 5 amyloid subtypes from liver heart and spleen. NTproBNP biomarker data from Phase 1b (100 ug/mL) were used, with GLS data (doses>100 mg/m² had effect): >30 ug/mL.

It was established that a loading dose to rapidly achieve C_{trough} of 130 ug/mL could be achieved by:
1 dose for 1500 mg/m²
2qw or 3 qw doses for 1000 mg/m² or 25 mg/kg
4qw doses for 750 mg/m² or 18.75 mg/kg
6qw doses for 500 mg/m² or 12.5 mg/kg,
   which covered 4qw doses for all dose levels including fixed doses.

Maintenance Dose Regimen

The maintenance dose regimen were established to:
cover q2w doses for all doses BSA and weight based
cover q4w for 1375 mg/m²
cover q3w and q4w for 1000 mg/m² or 25 mg/kg
cover q3w for 750 mg/m² or 18.75 mg/kg

Dose Regimen in Combination with PCD

It was further established that the antibody can be safely dose in combination with no impact on exposure. It was found ideal to dose antibody first and to modify the dose based on maintaining normal neutrophils and monocytes.

The hypothesis behind this was that a better organ response can be obtained by combination dosing of the antibody and doxycycline based on the mechanism of action. The antibody removes toxic light chains, protofibrils, fibrils and amyloid, and amyloid builds up in organs with extracellular matrix, while doxycycline inhibits matrix metalloproteases and prevents extracellular matrix allows greater access of the antibody to amyloid and N-terminus epitope.

The CARES clinical program consists of two parallel double-blind, randomized, event-driven global Phase 3 studies, which are evaluating the efficacy and safety of the antibody in AL amyloidosis patients who are newly diagnosed and naïve to standard of care (SoC) treatment (cyclophosphamide-bortezomib-dexamethasone (CyBorD) chemotherapy). One study is enrolling approximately 260 patients with Mayo stage IIIa disease [the antibody+CyBorD (n=178) and placebo+CyBorD (n=89)] and one study is enrolling approximately 110 patients with Mayo stage IIb disease [the antibody+CyBorD (n=74) and placebo+CyBorD (n=37)]. The studies will be conducted at approximately 70 sites across North America, the United Kingdom, Europe, Israel, Japan, and Australia. In each study, participants are being randomized in a 2:1 ratio to receive either the antibody plus SoC or placebo plus SoC once weekly for four weeks. This will be followed by a maintenance dose administered every two weeks until the last patient enrolled completes at least 50 weeks of treatment. Patients will continue follow-up visits every 12 weeks. The primary study objectives are overall survival and the safety and tolerability of the antibody. Key secondary objectives will assess functional improvement in the six-minute walk test (6MWT), quality of life measures (Kansas City Cardiomyopathy Questionnaire Overall Score & Short Form 36 version 2 Physical Component Score) and cardiac improvement (Global Longitudinal Strain, or GLS).

### Example 8

### Preliminary Results of the Liver Effects of the Antibody

Administration of the disclosed antibody showed improvement of liver function as measured by liver enzymes including: ALP, ALT, AST, GGT and bilirubin and size reduction of the liver showing the removal of amyloid and improved functioning. A reduction of at least 50% change in biomarker (liver enzymes) was associated with a response and improved liver functioning. Such effects on the liver were observed with antibody doses greater than 500 mg/m². The improvement in the liver function induced by the administration of the antibody were found to be irrespective of hematologic response.

### Example 9

### Preliminary Results of the Organ Response of the Antibody

Administration of the disclosed antibody showed some improvement of kidney function. 7 patients with kidney involvement and all had organ responses.

In particular, one patient with partial response (PR) subsequently progressed back to stable disease (SD). Despite this, the patient had an ongoing deepening renal organ response currently showing a 76% reduction in 24-hour proteinuria without change in anti-plasma cell therapy. The median was 56 days to organ response.

Administration of the disclosed antibody showed some Cardiac Response. 3/8 evaluable patients were newly diagnosed and their NT-proBNP has shown increases in the first 3 months of CyBorD therapy. One of 8 patients achieved cardiac organ response by NT-proBNP criteria.

### Example 10

The disclosed antibody dosed at 1000 mg/m² is the recommended dose in combination with CyBorD for the ongoing randomized, double blind Phase 3 trials described above. Organ responses particularly in the kidney were common even in relapsed patients. Only 1 patient is no longer on study due to need for change in anti-plasma cell therapy. Significantly, organ responses have been seen even without ongoing hematologic (partial response) PR.

### Example 11

### Phase 3 Study

Table 17 provides Demographics and Baseline Characteristics of a further Phase 3 study to assess dosage for the antibody of the invention having a heavy chain variable domain (VH) as set forth in SEQ ID NO:1 and a light chain variable domain (VL) as set forth in SEQ ID NO:2. The results show the range (min/max) and the median doses, e.g., the median dose was 25.6 mg/kg, ranging from 19.8 to 31.1 mg/kg, with the vast majority of patients receiving between 22 and 28 mg/kg.

TABLE 17

| | Stage IIIB | | Stage IIIA | | Total \| | |
|---|---|---|---|---|---|---|
| | (N = 16) | | (N = 52) | | (N = 68) | |
| Parameters | n (%) | | n (%) | | n (%) | |
| Age (yrs) | | | | | | |
| at consent | | | | | | |
| n | 15 | | 48 | | 63 | |
| Mean (SD) | 66.3 | (10.52) | 60.6 | (17.78) | 62.0 | (16.44) |
| Median | 68.0 | | 65.5 | | 66.0 | |
| Min, Max | 36.0, 78.0 | | 0.0, 81.0 | | 0.0, 81.0 | |
| Sex - n (%) | | | | | | |
| Male | 8 | (50.0) | 32 | (61.5) | 40 | (58.8) |
| Female | 7 | (43.8) | 16 | (30.8) | 23 | (33.8) |
| Missing | 1 | (6.3) | 4 | (7.7) | 5 | (7.4) |
| Race - n (%) | | | | | | |
| Black or | 1 | (6.3) | 4 | (7.7) | 5 | (7.4) |
| African | | | | | | |
| American | | | | | | |
| White or | 11 | (68.8) | 35 | (67.3) | 46 | (67.6) |
| Caucasian | | | | | | |
| Asian | 1 | (6.3) | 7 | (13.5) | 8 | (11.8) |
| Other | 2 | (12.5) | 2 | (3.8) | 4 | (5.9) |
| Missing | 1 | (6.3) | 4 | (7.7) | 5 | (7.4) |
| Ethnicity - n (%) | | | | | | |
| Hispanic or | 1 | (6.3) | 1 | (1.9) | 2 | (2.9) |
| Latino | | | | | | |
| Not Hispanic or | 13 | (1.3) | 43 | (82.7) | 56 | (82.4) |
| Latino | | | | | | |
| Unknown | 0 | (0.0) | 1 | (1.9) | 1 | (1.5) |
| Not Reported | 1 | (6.3) | 3 | (5.8) | 4 | (5.9) |
| Missing | 1 | (6.3) | 4 | (7.7) | 5 | (7.4) |
| Child-bearing | | | | | | |
| potential - n (%) | | | | | | |
| No | 14 | (87.5) | 48 | (92.3) | 62 | (91.2) |
| Yes | 1 | (6.3) | 0 | (0.0) | 1 | (1.5) |
| Missing | 1 | (6.3) | 4 | (7.7) | 5 | (7.4) |
| Parafin Embedded | | | | | | |
| Tissue Sample | | | | | | |
| Avaiable - n (%) | | | | | | |
| No | 11 | (68.8) | 37 | (71.2) | 48 | (70.6) |
| Yes | 3 | (18.8) | 9 | (17.3) | 12 | (17.6) |
| Missing | 2 | (12.5) | 6 | (11.5) | 8 | (11.8) |
| Weight (kg) | | | | | | |
| n | 15 | | 48 | | 63 | |
| Mean (SD) | 72.6 | (13.54) | 72.1 | (13.79) | 72.2 | (13.62) |
| Median | 71.4 | | 71.0 | | 71.0 | |
| Min, Max | 47.3, 95.9 | | 46.0, 111.6 | | 46.0, 111.6 | |
| Body Surface | | | | | | |
| Area (m^{A}2) | | | | | | |
| n | 16 | | 52 | | 68 | |
| Mean (SD) | 1.8 | (0.20) | 1.8 | (0.21) | 1.8 | (0.21) |
| Median | 1.8 | | 1.8 | | 1.8 | |
| Min, Max | 1.4, 2.2 | | 1.4, 2.3 | | 1.4, 2.3 | |
| Calculated Dose | | | | | | |
| Level (mg) | | | | | | |
| n | 16 | | 52 | | 68 | |
| Mean (SD) | 1841 | (202.8) | 1833 | (209.6) | 1835 | (206.5) |
| Median | 1800 | | 1825 | | 1815 | |
| Min, Max | 1440, 2150 | | 1420, 2270 | | 1420, 2270 | |
| Dose Level (mg/kg) | | | | | | |
| n | 15 | | 48 | | 63 | |
| Mean (SD) | 25.8 | (2.22) | 25.7 | (2.12) | 25.7 | (2.13) |
| Median | 25.5 | | 25.7 | | 25.6 | |
| Min, Max | 22.4, 30.4 | | 19.8, 31.1 | | 19.8, 31.1 | |
| Volume (mL) | | | | | | |
| n | 16 | | 52 | | 68 | |
| Mean (SD) | 61.4 | (6.76) | 61.1 | (6.99) | 61.2 | (6.88) |

| | | | |
|---|---|---|---|
| Median | 60.0 | 60.8 | 60.5 |
| Min, Max | 48.0, 71.7 | 47.3, 75.7 | 47.3, 75.7 |

Although the disclosure has been described with reference to the above examples, it will be understood that modifications and variations are encompassed within the spirit and scope of the disclosure. Accordingly, the disclosure is limited only by the following claims.

What is claimed is:

1. A pharmaceutical composition comprising:
(a) an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains;
(b) one or more isotonic agents;
(c) a buffer; and
(d) a non-ionic surfactant.

2. The composition of claim 1, wherein the antibody binds to kappa and lambda mis-folded light chains.

3. The composition of claim 1, wherein the isotonic agent is sodium acetate; the buffer is sodium chloride; the non-ionic surfactant is polysorbate 80.

4. The composition of claim 1, comprising from about 20 to 40 mg/mL antibody.

5. The composition of claim 1, comprising from about 15 to 35 mM sodium acetate.

6. The composition of claim 1, comprising from about 25 to 75 mM sodium chloride.

7. The composition of claim 1, comprising from about 0.5 to 5% mannitol.

8. The composition of claim 1, comprising from about 0.001 to 0.1% polysorbate 80.

9. The composition of claim 1, wherein the composition has a pH from about 5 to 6.

10. The composition of claim 9, wherein the composition has a pH of about 5.5.

11. The composition of claim 1, comprising:
30 mg/mL antibody;
about 25 mM sodium acetate;
about 50 mM sodium chloride;
about 1% mannitol; and
about 0.01%-0.05% polysorbate 80.

12. The composition of claim 1, wherein the antibody is a mixture comprising a native fraction, a reduced fraction, and/or a glycosylated or deglycosylated fraction, each having a heterogeneous charge.

13. The composition of claim 12, wherein the native fraction comprises sialylated species, neutral species, and/or galactosylated, fucosylated and/or mannosylated neutral species.

14. The composition of claim 12, wherein the reduced fraction comprises light chains with glycated lysines.

15. The composition of claim 1, wherein the antibody is a mixture comprising intact antibodies, halfmer fragments, incomplete antibody fragments, other fragments and/or aggregates thereof.

16. The composition of claim 15, wherein the halfmer is an antibody comprising one or two heavy chains (HC) and one light chain (LC).

17. The composition of claim 15, wherein the incomplete antibody is missing a C-terminal region of a HC.

18. The composition of claim 15, wherein fragments comprise HC retaining C-terminal lysine.

19. A method of decreasing an amount of amyloid deposits in a subject comprising administering to the subject about 500 to 1,000 mg/m² of an antibody having a heavy chain variable domain (VH) having an amino acid sequence of SEQ ID NO:1 and a light chain variable domain (VL) having an amino acid sequence of SEQ ID NO:2, thereby decreasing the amount of amyloid deposits in the subject.

20. The method of claim 19, wherein the antibody is administered weekly for at least 2, 3, or 4 weeks.

21. The method of claim 20, further comprising administering a maintenance dose of antibody to the subject thereafter.

22. The method of claim 21, wherein the maintenance dose is administered biweekly, triweekly, or monthly after the first 2, 3, 4 or more weeks.

23. The method of claim 19, wherein administering a weekly dose of about 1,000 mg/m² of antibody comprises administering about 20-25 mg/kg of antibody.

24. The method of claim 19, wherein administering a weekly dose of about 1,000 mg/m² the antibody comprises administering about 2,750 mg of the antibody.

25. The method of claim 19, wherein the subject is newly diagnosed with an amyloidosis disease or disorder prior to administration of the antibody.

26. The method of claim 19, wherein the subject has been previously treated for an amyloidosis disease or disorder prior to administration of the antibody.

27. The method of claim 19, wherein the amyloidosis disease or disorder is selected from the group consisting of light chain (AL) amyloidosis, autoimmune (AA) amyloidosis and hereditary (TTR) amyloidosis.

28. The method of claim 19, wherein administering the antibody comprises administering to the subject a pharmaceutical composition of any of claims 1-18.

29. A method of treating an amyloidosis disease or disorder in a subject comprising administering to the subject:
(a) the pharmaceutical composition of any of claims 1-18; and
(b) an additional therapy,
thereby treating the amyloidosis disease or disorder in the subject.

30. The method of claim 29, wherein the antibody administration dose is from about 500 mg/m² to 1,000 mg/m².

31. The method of claim 30, wherein the dose is selected from about 500 mg/m², about 750 mg/m² and about 1,000 mg/m².

32. The method of claim 30, wherein a weekly dose of about 500 mg/m² of antibody comprises about 12.5 mg/kg of antibody, a weekly dose of about 750 mg/m² comprises about 18.75 mg/kg of antibody and a weekly dose of about 1,000 mg/m² of antibody comprises about 25 mg/kg of antibody.

33. The method of claim 30, wherein administering about 500 mg/m² of antibody comprises administering about 1,375 mg of antibody, administering about 750 mg/m² of antibody comprises administering about 2,065 mg of antibody, and administering about 1,000 mg/m² of antibody comprises administering about 2,750 mg of antibody.

34. The method of claim 30, wherein the 500 mg/m², 750 mg/m² and 1,000 mg/m² administration dose achieves a site occupancy of a target receptor of at least 90%.

35. The method of claim 29, wherein the antibody is administered weekly for at least 2, 3 or 4 weeks.

36. The method of claim 35, further comprising administering a maintenance dose of antibody to the subject thereafter.

37. The method of claim 36, wherein the maintenance dose is administered biweekly, triweekly, or monthly after the first 2, 3, 4 or more weeks.

38. The method of claim 29, wherein the subject has a hematologic disease.

39. The method of claim 38, wherein the hematologic disease is characterized by an involved/uninvolved free light chain difference (dFLC) >5 mg/dL or by a FLC >5 mg with abnormal ratio.

40. The method of claim 38, wherein the hematologic disease is characterized by a serum protein electrophoresis (SPEP) or a urine protein electrophoresis (UPEP) m-spike >0.5 g/dL.

41. The method of claim 29, wherein the antibody is administered prior to, simultaneously with, or after the additional therapy.

42. The method of claim 41, wherein the antibody is administered prior to the additional therapy.

43. The method of claim 29, wherein the additional therapy comprises cyclophosphamide, bortezomib, dexamethasone, daratumumab, melphalan, lenalidomide, isatuximab, venetoclax, a stem cell transplant or a combination thereof.

44. The method of claim 29, wherein the antibody is administered by intravenous (IV) infusion, subcutaneous injection or intramuscular injection.

45. The method of claim 29, wherein the subject is newly diagnosed with an amyloidosis disease or disorder prior to administration of the antibody.

46. The method of claim 29, wherein the subject has been previously treated for an amyloidosis disease or disorder prior to administration of the antibody.

47. The method of claim 29, wherein the amyloidosis disease or disorder is selected from the group consisting of light chain (AL) amyloidosis, autoimmune (AA) amyloidosis and hereditary (TTR) amyloidosis.

48. The method of claim 29, wherein the amyloid deposits comprise aggregates of λ-light chain fibrils and/or κ-light chain fibrils.

49. The method of claim 29, wherein administering the pharmaceutical composition induces removal of amyloid deposits present in an organ or tissue.

50. The method of claim 49, wherein the organ or tissue is selected from the group consisting of heart, kidney, liver, lung, gastrointestinal tract, nervous system, muscular skeletal system, soft tissue, skin and any combination thereof.

51. A method of treating an amyloidosis disease or disorder involving a kidney, a gastrointestinal tract, liver or a heart in a subject comprising administering to the subject the composition of any of claims 1-18;
thereby treating the amyloidosis disease or disorder in the subject.

52. The method of claim 51, wherein the antibody dose is from about 500 mg/m² to 1,000 mg/m².

53. The method of claim 51, wherein the antibody dose is selected from about 500 mg/m², about 750 mg/m² and about 1,000 mg/m².

54. The method of claim 51, wherein the antibody is administered weekly for at least 2, 3 or 4 weeks.

55. The method of claim 54, further comprising administering a maintenance dose of the antibody to the subject thereafter.

56. The method of claim 55, wherein the maintenance dose is administered biweekly, triweekly, or monthly after the first 2, 3, 4 or more weeks.

57. The method of claim 51, further comprising administering to the subject an additional therapy.

58. The method of claim 57, wherein the additional therapy comprises cyclophosphamide, bortezomib, dexamethasone, daratumumab, melphalan, lenalidomide, isatuximab, venetoclax, a stem cell transplant or a combination thereof.

59. A method of treating one or more symptoms of an amyloidosis disease or disorder involving the skin in a subject comprising administering to the subject the pharmaceutical composition of any of claims 1-18, thereby treating the skin in the subject.

60. The method of claim 59, wherein an antibody dose is from about 500 mg/m² to 1,000 mg/m².

61. The method of claim 60, wherein the antibody dose is selected from about 500 mg/m², about 750 mg/m² and about 1,000 mg/m².

62. The method of claim 59, wherein the one or more symptoms of the amyloidosis disease or disorder involving the skin comprise hair loss, body hair loss and facial hair loss.

63. The method of claim 59, wherein the antibody is administered weekly for at least 2, 3 or 4 weeks.

64. The method of claim 63, further comprising administering a maintenance dose of the antibody to the subject thereafter.

65. The method of claim 64, wherein the maintenance dose is administered biweekly, triweekly, or monthly after the first 2, 3, 4 or more weeks.

66. The method of claim 59, further comprising administering to the subject an additional therapy.

67. The method of claim 66, wherein the additional therapy comprises cyclophosphamide, bortezomib, dexamethasone, daratumumab, melphalan, lenalidomide, isatuximab, venetoclax, a stem cell transplant or a combination thereof.

68. A method of inhibiting and/or reducing aggregation of light chains and amyloid fibrils in a subject comprising administering to the subject about 500 mg/m² to 1,000 mg/m² of an antibody having a heavy chain variable domain (VH) having an amino acid sequence of SEQ ID NO:1 and a light chain variable domain (VL) having an amino acid sequence of SEQ ID NO:2; thereby inhibiting and/or reducing the aggregation of light chains and amyloid fibrils in the subject.

69. The method of claim 68, wherein an antibody dose is about 1,000 mg/m².

70. The method of claim 68, wherein the antibody dose is selected from about 500 mg/m², about 750 mg/m² and about 1,000 mg/m².

71. The method of claim 68, wherein the antibody is administered weekly for at least 2, 3 or 4 weeks.

72. The method of claim 70, further comprising administering a maintenance dose of the antibody to the subject thereafter.

73. The method of claim 72, wherein the maintenance dose is administered biweekly, triweekly, or monthly after the first 2, 3, 4 or more weeks.

74. A method of treating an amyloidosis disease or disorder in a subject comprising administering to the subject:
the pharmaceutical composition of any of claims 1-18; thereby treating the amyloidosis disease or disorder in the subject.

75. The method of claim 74, wherein administering the pharmaceutical composition renders the subject eligible for a stem cell transplant.

76. The method of claim 74, further comprising performing a stem cell transplant in the subject.

77. The method of claim 74, wherein an antibody dose is from about 500 mg/m² to 1,000 mg/m².

78. The method of claim 77, wherein the antibody dose is selected from about 500 mg/m², about 750 mg/m² and about 1,000 mg/m².

79. The method of claim 74, wherein the antibody is administered weekly for at least 2, 3 or 4 weeks.

80. The method of claim 79, further comprising administering a maintenance dose of the antibody to the subject thereafter.

81. The method of claim 80, wherein the maintenance dose is administered biweekly, triweekly, or monthly after the first 2, 3, 4 or more weeks.

82. A method of treating amyloidosis in a subject having multiple myeloma comprising administering to the subject a pharmaceutical composition comprising an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains,
thereby treating amyloidosis in the subject.

83. The method of claim 82, wherein the antibody binds to kappa and lambda mis-folded light chains.

84. The method of claim 82, wherein the pharmaceutical composition further comprises:
(a) one or more isotonic agents;
(b) a buffer; and
(c) a non-ionic surfactant.

85. The method of claim 84, wherein the isotonic agent is sodium acetate; the buffer is sodium chloride; and the non-ionic surfactant is polysorbate 80.

86. The method of claim 82, wherein the pharmaceutical composition comprises:
30 mg/mL antibody;
about 25 mM sodium acetate;
about 50 mM sodium chloride;
about 1% mannitol; and
about 0.01%-0.05% polysorbate 80.

87. The method of claim 82, wherein the antibody administration dose is from about 500 mg/m² to 1,000 mg/m².

88. The method of claim 87, wherein the dose is selected from about 500 mg/m², about 750 mg/m² and about 1,000 mg/m².

89. The method of claim 88, wherein a weekly dose of about 500 mg/m² of antibody comprises about 12.5 mg/kg of antibody, a weekly dose of about 750 mg/m² comprises about 18.75 mg/kg of antibody and a weekly dose of about 1,000 mg/m² of antibody comprises about 25 mg/kg of antibody.

90. The method of claim 88, wherein administering about 500 mg/m² of antibody comprises administering about 1,375 mg of antibody, administering about 750 mg/m² of antibody comprises administering about 2,065 mg of antibody, and administering about 1,000 mg/m² of antibody comprises administering about 2,750 mg of antibody.

91. The method of claim 88, wherein the 500 mg/m², 750 mg/m² and 1,000 mg/m² administration dose achieves a site occupancy of a target of at least 90%.

92. The method of claim 86, wherein the antibody is administered weekly for at least 2, 3 or 4 weeks.

93. The method of claim 92, further comprising administering a maintenance dose of antibody to the subject thereafter.

94. The method of claim 93, wherein the maintenance dose is administered biweekly, triweekly, or monthly after the first 2, 3, 4 or more weeks.

95. The method of claim 82, wherein the pharmaceutical composition is administered by intravenous (IV) infusion, subcutaneous injection, or intramuscular injection.

96. The method of claim 82, wherein the subject is currently or has been previously treated for multiple myeloma.

97. The method of claim 96, wherein a treatment for multiple myeloma is selected from the group consisting of chemotherapy, corticosteroid, immunomodulating agent, proteasome inhibitor, histone deacetylase (HDCA) inhibitor, immunotherapy, nuclear export inhibitor, stem cell transplant, radiation therapy, surgery, and any combination thereof.

98. A method of treating amyloidosis in a subject having a plasma cell disease comprising administering to the subject a pharmaceutical composition comprising an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains,
thereby treating the plasma cell disease in the subject.

99. The method of claim 98, wherein the plasma cell disease is selected from the group consisting of low-grade B-cell lymphoma, monoclonal gammopathy of undetermined significance (MGUS), and multiple myeloma.

100. The method of claim 98, wherein the antibody administration dose is from about 500 mg/m² to 1,000 mg/m².

101. The method of claim 100, wherein the dose is selected from about 500 mg/m², about 750 mg/m² and about 1,000 mg/m².

102. The method of claim 100, wherein the antibody is administered weekly for at least 2, 3 or 4 weeks.

103. The method of claim 100, further comprising administering a maintenance dose of antibody to the subject thereafter.

104. The method of claim 100, wherein the pharmaceutical composition is administered by intravenous (IV) infusion, subcutaneous injection, or intramuscular injection.

105. The method of claim 100, wherein the subject is currently or has been previously treated for the plasma cell disease.

106. The method of claim 105, wherein a treatment for plasma cell disease includes chemotherapy.

107. A method of identifying a subject having multiple myeloma as a candidate for an anti-amyloidosis treatment comprising identifying light chain (AL) amyloidosis fibrils and/or amyloid protein precursor deposition in the subject,
wherein the identification of AL amyloidosis fibrils and/or amyloid protein precursor deposition in the subject is indicative of the likelihood of the subject to respond to the anti-amyloidosis treatment, and
wherein the anti-amyloidosis treatment comprises an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains,
thereby identifying the subject as a candidate for the anti-amyloidosis treatment.

108. The method of claim 107, wherein the subject is currently or has been previously treated for multiple myeloma.

109. The method of claim 107, further comprising administering to the subject a treatment for multiple myeloma.

110. The method of claim 108 or 109, wherein a treatment for multiple myeloma is selected from the group consisting of chemotherapy, corticosteroid, immunomodulating agent, proteasome inhibitor, histone deacetylase (HDCA) inhibitor, immunotherapy, nuclear export inhibitor, stem cell transplant, radiation therapy, surgery, and any combination thereof.

111. A method of treating amyloidosis in a subject having a B-cell lymphoproliferative disorder comprising administering to the subject a pharmaceutical composition comprising an antibody having a heavy chain variable domain (VH) having an amino acid sequence as set forth in SEQ ID NO:1 and a light chain variable domain (VL) having an amino acid sequence as set forth in SEQ ID NO:2 and that binds to light chains,
thereby treating the amyloidosis in the subject.

112. The method of claim 111, wherein the B-cell lymphoproliferative disorder is selected from the group consisting of multiple myeloma, Waldenstrom's macroglobulinemia, chronic lymphocytic leukemia, non-Hodgkin lymphoma and lymphomas associated with amyloidosis.

113. The method of claim 111, wherein the B-cell lymphoproliferative disorder is multiple myeloma.

114. The method of claim 111, wherein the antibody administration dose is from about 500 mg/m² to 1,000 mg/m².

115. The method of claim 114, wherein the dose is selected from about 500 mg/m², about 750 mg/m² and about 1,000 mg/m².

116. The method of claim 114, wherein the antibody is administered weekly for at least 2, 3 or 4 weeks.

117. The method of claim 114, further comprising administering a maintenance dose of antibody to the subject thereafter.

118. The method of claim 114, wherein the pharmaceutical composition is administered by intravenous (IV) infusion, subcutaneous injection, or intramuscular injection.

119. The method of claim 114, wherein the subject is currently or has been previously treated for the B-cell lymphoproliferative disorder.

120. The method of claim 119, wherein a treatment for B-cell lymphoproliferative disorder includes chemotherapy.

121. The method of any of claims 29, 51, 59, 68, 82, 107 or 111, wherein the dose of antibody is from about 20 mg/kg to 31 mg/kg.

121. The method of any of claims 29, 51, 59, 68, 82, 107 or 111, wherein the dose of antibody is from about 22 mg/kg to 28 mg/kg.


SEQUENCE LISTING (10 sequences)

SEQ ID NO: 1 - protein, 111 aa, Artificial Sequence
  Description of Artificial Sequence Synthetic polypeptide
QVQLKESGPGLVAPSQSLSITCTVSGFSLSSYGVSWVRQPPGKGLEWLGVIWGDGSTNYHPNLMSRLSISKDISKSQVLFKLNSLQTDDTATYYCVTLDYWGQGTSVTVSS

SEQ ID NO: 2 - protein, 112 aa, Artificial Sequence
  Description of Artificial Sequence Synthetic polypeptide
DVVMTQTPLSLPVSLGDQASISCRSSQSLVHRNGNTYLHWYLQKPGQSPKLLIYKVSNRFSGVPDRFSGSGSGTDFTLKISRVEAEDLGLYFCFQTTYVPNTFGGGTKLEIK

SEQ ID NO: 3 - protein, 17 aa, Artificial Sequence
  Description of Artificial Sequence Synthetic peptide
SSQSLVHRNGNTYLHWY

SEQ ID NO: 4 - protein, 6 aa, Artificial Sequence
  Description of Artificial Sequence Synthetic peptide
KVSNRF

SEQ ID NO: 5 - protein, 6 aa, Artificial Sequence
  Description of Artificial Sequence Synthetic peptide
QTTYVP

SEQ ID NO: 6 - protein, 7 aa, Artificial Sequence
  Description of Artificial Sequence Synthetic peptide
SYGVSWV

SEQ ID NO: 7 - protein, 12 aa, Artificial Sequence
  Description of Artificial Sequence Synthetic peptide
PNLMSRLSISKD

SEQ ID NO: 8 - protein, 6 aa, Artificial Sequence
  Description of Artificial Sequence Synthetic peptide
DYWGQG

SEQ ID NO: 9 - protein, 441 aa, Artificial Sequence
  Description of Artificial Sequence Synthetic polypeptide
QVQLKESGPGLVAPSQSLSITCTVSGFSLSSYGVSWVRQPPGKGLEWLGVIWGDGSTNYHPNLMSRLSISKDISKSQVLFKLNSLQTDDTATYYCVTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, Artificial Sequence
  Description of Artificial Sequence Synthetic polypeptide
DVVMTQTPLSLPVSLGDQASISCRSSQSLVHRNGNTYLHWYLQKPGQSPKLLIYKVSNRFSGVPDRFSGSGSGTDFTLKISRVEAEDLGLYFCFQTTYVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC